SEQUENCE LISTING (36 sequences)

SEQ ID NO: 1 - DNA, 10046 bp, Solanum sparsipilum
  promoter (1)..(1657)
  5′UTR (1658)..(1821)
  CDS (1822)..(2330)
  Intron (2331)..(2525)
  CDS (2526)..(3615)
  Intron (3616)..(4226)
  CDS (4227)..(4532)
  Intron (4533)..(6843)
  CDS (6844)..(8322)
  3′UTR (8326)..(8331)
  Intron (8332)..(8464)
  3′UTR (8465)..(8811)
gtggagcatgggttttcgttatctttatcggagatgttgaaggttagtgatggggaattcaatgccagattgattgaaatgcgattgaggatagtggaagataagcaattgtagtaagtaagcaattcttttctatgagtagagtcgatttttgtatatgttggagaatcattagagttaatactaaatgttttaacatatattcattgtggttagatgtacatttttgcgataaagtaatgatttttatccttgatgtggtcttcttcatggtttttattgtttctcgtggaatgcaattcgaagtttgctatgctcgtgttgtgttgtttaagtattaatttagtatgcttgcaactttgcaagggttagttcattggcattggcgatgatgcataagtttaatggttgaatgactatgttatacgttcgaataattgaagtatgctagaaacttgttatgcgagagttctgttgcattctcttttctcatgctgaaacaatggttcaacaacgggtagtaagcaattaataacacatatgtccatgctttaattgttcctttctataacacctcgtttgacattgctcagctttctgccggccagagatttgtgcaactattacatttctataagtagctcgatcccagactggttctactgaaagatgtgcttctccacgtctgtagaaaatcgttagactttgttgttgacatattcttgttggtattccgttatctgactctatagatactcgaaatcttatgtctagggcattcttctataatatcaaacatgtactttcgaagcttgaaggttgctattgtagtttcctccttgctgttgaaccttgggaagagttgttatctaccagtcaggtgcttctgatatatgagcgttaaagttgggctagtgttttgccctttgttaaacctcaaattatgttttgttccaattgaacacataatgaagtcttctcataaattttatgctaaattgattttatggtatactacattttattagaacaagctttgaggagctatattacatactcaaatatatatactgcatatgtttttttttttatgcaaataacaccattaatattgatcgtcactctctcgtttgccaaaaaaaatatttataccatacatgtatatggtaatttatgcataaattttataaaatgacaagtattgtgatccagctatttacagaaagagatgacatataaaatgagacgcagggagtattttttattattaatatgtaaacaataaaccagaaaataaggaggaaaagaaaggaaaaaaacatttatagactataaatgaaggagaatatgaatgtcatttaactcttattaatcgtgaacattaaagtactattgcttcatttcaaatcacctacccgtttaattatgattttataatattgagggtaaaagtaaatattcataaactactccctccattccatattaatgagatatttttcatttttcaaattcggacctttgttttttaatcaaatttttgtggcctgctggctattttattcaactttctgaaaccaacatttagtacaaggactttggcccaattaaaaaataccaaaacaaaataaaattaatctagaagatccagcccaacaaatatcaggttctagggcatttccacatttgtaaagtgaatggacaacagccatatcctccgccgccttgtctgaattgtcaatacaaggactttgtccaaaatatcatcaattgatctcatccataaaaaatatttttcttttcagacaattaatcgaatctatggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggttagttgaatacacataattacttttaatgaaaaaatggaatcatttccattcaaacacaatataatttcattgattattatttagatggtagagtatatacatttttattgttaagaaggcatagttctatcaatttaattagagaggatacataaaagtcctcctaaactatcaatcttcttctttatgtaggattgaatcagagcatattcaacagatcgtagactacatctattccaaattttgcacaaatgcttattcttcatcttttttgcaaggtgttgtgggaataaacgatcacttagagaaactaaaatccaaacttcaaatggaaatcaacgatgttcggattttagggatatggggaataggcggagtcggtaaaacgacaatagcaaatgccatttttgatactatatcttatcaatttaaagcttcctgttttcttgcagatgttaaagaaaatgcaaaaaagaatgaactgtattctttacaaaatacccttctctctgaactgttaagaaaaaaagatgattatgtcaataataagtatgctgggaagcgcatgattccgagcatactttggtctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaggctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaacaactgtagagcaaataaagaaaaactctaattcagaaattgttgaaaaactcaaaataagttatgatgggttggagctcgaagagcaagagatatttctagatattgcatgtttcttacgtgaaattgaaagaaaagaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagtttccgaaaaaacgtagcagaatatgggatgctgaagatttcaaaaaagtgatgatagactatacagtaagtaagctaaacaatgcaataatgtttaatttctaatttttatatttcaaagacgtataagccaatcaattccaattatttgttcctcttgcttcatattcttacaggtacgtcattttagctctttactttactttatttatttatttaatttttaataaaagaagcaaaagtaacatcaattgcctaacatagctaatcgtctatgaattagtgtttaatatgttttatccagtttctttcttaagattgcatgttatacacaccaataagtaaatagtttttaaagctaatatcacccacatatttaccctataaaaatcctcatataaaaaaatacaagtaagaagggtcaaatcataacataccaatcctcaacaaagaattcacttaatcattgatttaaaaataaataaattaaaagatgacaattatttgtattgttccaattacaatatttattctatcactacattgaattttttatgatttgatgctaaagaacttctaaaaaaaagtattattagaaaatataaatatttgcacaaaatattttttgtaagcaacatgtgaagttatgattagttgactcatgatctttttatcaggggaccatgacagtggaagcaatctggtttaggtgctataaagaagaactatactttaataatgaggcaatggaaaaaatgaaaagtcttaggatattacaagttgatggttataacaaattctttgcttcacgtccctcttcgaatcaccatgatgactctattgagcacatgtccaataacttgcgttggttagtctggaatcactattcttggaagtcattgccagaatattttaaaccagaaaagcttgtacatcttgaacttcgtaacagttcgctgcattatttatggaaggaaacaaaggtaccattttatttaagctactttctaaggaaaagggtaaatacccctcaactttgtgatttgaaactgatatatcctttgttttaaaaaatgatatatatactctgtcgtctaacaaatgatgcatatgtaccatttccgttaacaggtctgtatttattgaattaaaaaaataattataaaattgatttttaaaattcaaaaatatcacgtggctttaaaaaattaccccactcatttgttttcctctctagacccaacccaaatgaataaaaacccaatccatcttctactcaaacataagatgagtttgagtcggatctgaataaagagggatgaggttctttagttggattaggtctagaggggtaagtgagtaatttttttaaaaaccatgtgagtgtttagaaattaaaaaactaacttcaaattttttaaaatttaattgttaatgaaaataataaatatgcattatttgttaaacgttcatgttatatatgcatcattttatgttgttaacaaaagagatcacatagttaagatgtatatctgccccttttttctactttctatggttacttgtcctataattatggtgtttgtatttaaatgaaacaggttattaattccataaactcaaattaatatatgttatacaatattgagttttcatattgttactatgaaaaagggctaaaattgtccttaaactaacaagtatagctcaattaattcttcatttgcttctgacaaccaaaacaccctgccatctattttctgatggatttaatgtaacatacacttttaaacaatgaagttaagttcgagtttaaaaaaatagattaggtaagaatcactcattgatacgacaaaacccaacatcatataactgtaaaaattaaataagcaatttcttcaaaggcaatttaaccttattttgtcatattgaattgtcatgtcagctttttctattatttaaatggccctaacatgctcatgtggacaacattagcttggaggcattttttggtccaaaaaatatatagcaagggtatttttggtataccaaaagcaaatgaaggataaaatggagctatttctgatagttcaaggacaaattaatcccttttctattaaaattataatatcgttatgtcttttttttcaaagcttgcctcattattttaactaagaagctcaatttaaaatttgagcattcaaatatttacctacaaaaatatgaatctcatcctaggttttatgtagtgggacaaaatgaatacttatcgaaggagttcattctatactactatcttcttgaatgatgattaaggtttcatatattggatttgggctaaaactataccatatctataggaataggttcattttggtcttcaaatatatataatatgaacatttttaatcccttcattttgctaaagtggagtacgtttaatctccttaataaaatccgtttaaaagtaacagtgttaaactcaagtaacatccacatgactcttaaaacataaaatccatcgggtaaactcattccccgtttaatagttgtctagatgccatattcttaatatttgatctacaccgacaactaaacactctcaccaaaagaaaaaaaaaagataattaaaataaatagtcattacagctaaaggattaaaaaaaacataaattggggttaaaattatgccctccatttttcttttcactcaagcacctagaagagtgtattcactcttttccacatcaatgttcaagagataaatgatactccctccgtttcacaaaaaatgacctcttttttgttttactcagtttaaaaaagaatgacctttttctatttttggtaacattttaatttcagtttttcacgtgacatgtttaaggccacaagattaaaggacaattttgtacatttgacataactttaacgtaggaccacagattcaaaagtcttctttattttcttaaacgtcatgtcaagtcaaactagaccattctttgtgaaatggagagagtagtaaatagtcaagtaaatataggggatatttaagataagtgtaacaatcaagtgtgtatcgaataaaacatgtcaagtctaaaggattatagataataaacacttttatttagtttctgaacatgtaatttccatataaaaataaaataaaacattttacaatttcactcaaaatattgtgttagactctcataattcagatgtctttattcatttttggtctaaggggtaaattaaacacaaagatcattctttctctattttgaatagcagcatttgccgtctctacgaaagctagatctcagctattctgaaagcctggtgcaaacaccagatttcacggggatgccaaatttggagtatttgactctggagggctgtagaaagcttgaagaggttcactattccctagcatgttgcgaaaaactcattgagttaaatttgtgtttgtgtttcaagcttaggagatttccatgggttaataacatgaaatctatgaatctacgatgttgctatagtttaatggaatttccagaattcctcggtacaatgaagccagagttagtgagtctcaaagtaaacagtaggataagggaactaccattatctattcagtacctaattcatctaacagagctagatttgagaaacatggaaacccttgaagctcttccaagcagcattggtaagttgaaaggtttggtgaagctaaatgtgtcgcgctgcttcacaattaaaagcttgcccgaagagataggtgatttagaaaacttggaggaacttgatgcttcatatactctaatttcacgacctccgtcttccattgtccgcttgaacaagcttaaatacttgaagtttgtaagaatgaaaacagaagatgaagtgtactttgtgtttcctccaattaatggcgggttactctcattggaaatcctggagctcggttcctccaaattcatagatggaagaattccagaagatattggatacttatcctctttgaaagatttgcatctcgagggagataattttgagcatttgcctcaaagcatagcccaacttggtgctcttcggttcttgcacttagtacgttgcacgaggcttacacagctgccagaatttccaccacaattagatacaatatatgcagattggcgcaatgatttgatctgtaattcactgtttcaaaatatctcatcattccagcatgacatctctgcttcagattccttgtcgttaagagtgtttacgagttcggggagtaatataccaagttggttccactatcagggaatggataaaagtgtttcagtcaatttgcctgaaaactggtatgtatcagataacttcttgggatttgctgtatgttactctggaagtttaattgaaaactcggctcaattgattattagttctgaagggatgccgtgtatcacccagaaacttgtcttatccaatcattcagaatataaatatttcaagtttcggtttttcttggtaccttttgctggcatatgggatacatctaacgcaaatggtaaaacaccgaatgactatgggcacattatgttatcttttcctgaagaattggagggatgtggacttcgtttgttctataaagatgaatctgagcttgttgagaccaatgatgaaccatcaacagaactttcccttgggataaggaggatcagatacgacgatagtgaacatcatgaagaagccagttgttcatcttctaagaaacaaaggtcataagtagaggtatatatctcgatatgtatctaaacttctcgcttagctttctgttattaatagttcattttgatgctctttgtggttattataatagaggaaatttttgttctaaatacaaatatctctcatgttatggcagggaggttggaaaaacgatgaagcattaatctaagtgtgagacagtggaggaagtagtttgtgcatacactgcggatgatcaatctagcacgtggattaagtagctcagatcgagcaattacattgttttttcatttagtgtctggtacctgtcatagggtttccacttaatgtaattcaaatgccacactgttggaaccaattgacttaagccaattaaaaattattttgtcaatcccttgttaccgatcaaatgttgtgctttaggaagttgtgaaaagggaaaatgaacaaatgatcccttgtgatttttttttatttgaataaacataatgaaagtttagccaattgacaagtttaagaggttttagttattttaccttttatttataataataataatacttttatttagtttcttgggtgtgcaatagtttgagaaacagttacattttccatttttcatttcgaaaatataatcacattttccatttgctaaagtaactggtccaaaataaatcaaattgcagatgaatatatggtaagataattttgcgaaaacgggtggatgaaagaagcctcggaactgtttctagctgaccatccaaatgccttaattgggtgttcatttgagtaagttttaggttttaaaaacataaaaatcatattagcgctctatagacagttttgctatttgcgtttcatagcaaattatatgccgtttgtataaaagcgcaacgattgtatatgtatatcgattaaataattatatatatgtaactactatgtatatgtatcaattattgtttttgtatatctgcataaaatttaaatttgtatgcaattgaatcgaaataaaacatttgtatatcaaacatctctcgctttatacaatacaaattatacattgtcattgtattgtgtttgtataaagcaagaaagagagaaaggcaaaagagaactggtagggaaagaattgtatttatataattataagtgtataggacgaaaatatatttgcatttgtatttgtatatacaattttttcttgtttatacaaaaacaaacacaatttatacatttgtattgtataaaatgagagaggcgagagagagtggcgagagagatttcttgggagagaggctaatggcaaagtgtttgctacgaattagaatttaatgaaactatagttataatatttatttcgaattaatagtttgttatttcatacaatttccctataaagtatgataaatagctggccttccttagatttatgggccttttgaagatgtgtcaacgttgctggcctcttgggcttttaaggcccaagaaggtgggctttctgctaggaaatgaagtagttaaagctaaaagatttgtgtcggttatatatgtagttaatatgtagatcggatatattaaatattggcttgaatacattaaagaaataagagattttaaaggtttttagaaatagaaggggatattggaaataaggaaaacataagacgtgtatttcaataatttttcctagttaatatatcatgatctgttgagttatgggctttaagggagtgaat

SEQ ID NO: 2 - protein, 1128 aa, Solanum sparsipilum
MASSSSSASNLKYCPRWKYVVFLSFRGEDTRKTFTGHLYEGLKNRGISTFQDDKRLEHGDSIPKELLRAIEESQVALIVFSKNYATSRWCLNELVKIMECKDEENGQTVIPIFYDVDPSHVRNQSESFGAAFAEHELKYKDDVEGMQKVQRWRNALTVAANLKGYDIRDRIESEHIQQIVDYIYSKFCTNAYSSSFLQGVVGINDHLEKLKSKLQMEINDVRILGIWGIGGVGKTTIANAIFDTISYQFKASCFLADVKENAKKNELYSLQNTLLSELLRKKDDYVNNKYAGKRMIPSILWSMKVLIVLDDIDHSEHLEYLAGDVDWFGNGSRVIVTTRNKHLIEKDDAIYEVSTLPDHEAMQLFNKHAFKKEDPDESFKKFSLEVVNHAKGLPLALKVWGSLLHKKCLTLWRTTVEQIKKNSNSEIVEKLKISYDGLELEEQEIFLDIACFLREIERKEVMQILESCDFGAEYGLNVLINKSLVFISENDRIEMHDLIEDMGRYVVKMQKFPKKRSRIWDAEDFKKVMIDYTGTMTVEAIWFRCYKEELYFNNEAMEKMKSLRILQVDGYNKFFASRPSSNHHDDSIEHMSNNLRWLVWNHYSWKSLPEYFKPEKLVHLELRNSSLHYLWKETKHLPSLRKLDLSYSESLVQTPDFTGMPNLEYLTLEGCRKLEEVHYSLACCEKLIELNLCLCFKLRRFPWVNNMKSMNLRCCYSLMEFPEFLGTMKPELVSLKVNSRIRELPLSIQYLIHLTELDLRNMETLEALPSSIGKLKGLVKLNVSRCFTIKSLPEEIGDLENLEELDASYTLISRPPSSIVRLNKLKYLKFVRMKTEDEVYFVFPPINGGLLSLEILELGSSKFIDGRIPEDIGYLSSLKDLHLEGDNFEHLPQSIAQLGALRFLHLVRCTRLTQLPEFPPQLDTIYADWRNDLICNSLFQNISSFQHDISASDSLSLRVFTSSGSNIPSWFHYQGMDKSVSVNLPENWYVSDNFLGFAVCYSGSLIENSAQLIISSEGMPCITQKLVLSNHSEYKYFKFRFFLVPFAGIWDTSNANGKTPNDYGHIMLSFPEELEGCGLRLFYKDESELVETNDEPSTELSLGIRRIRYDDSEHHEEASCSSSKKQRS

SEQ ID NO: 3 - DNA, 3387 bp, Solanum sparsipilum
  CDS (1)..(3387)
atggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggattgaatcagagcatattcaacagatcgtagactacatctattccaaattttgcacaaatgcttattcttcatcttttttgcaaggtgttgtgggaataaacgatcacttagagaaactaaaatccaaacttcaaatggaaatcaacgatgttcggattttagggatatggggaataggcggagtcggtaaaacgacaatagcaaatgccatttttgatactatatcttatcaatttaaagcttcctgttttcttgcagatgttaaagaaaatgcaaaaaagaatgaactgtattctttacaaaatacccttctctctgaactgttaagaaaaaaagatgattatgtcaataataagtatgctgggaagcgcatgattccgagcatactttggtctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaggctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaacaactgtagagcaaataaagaaaaactctaattcagaaattgttgaaaaactcaaaataagttatgatgggttggagctcgaagagcaagagatatttctagatattgcatgtttcttacgtgaaattgaaagaaaagaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagtttccgaaaaaacgtagcagaatatgggatgctgaagatttcaaaaaagtgatgatagactatacagggaccatgacagtggaagcaatctggtttaggtgctataaagaagaactatactttaataatgaggcaatggaaaaaatgaaaagtcttaggatattacaagttgatggttataacaaattctttgcttcacgtccctcttcgaatcaccatgatgactctattgagcacatgtccaataacttgcgttggttagtctggaatcactattcttggaagtcattgccagaatattttaaaccagaaaagcttgtacatcttgaacttcgtaacagttcgctgcattatttatggaaggaaacaaagcatttgccgtctctacgaaagctagatctcagctattctgaaagcctggtgcaaacaccagatttcacggggatgccaaatttggagtatttgactctggagggctgtagaaagcttgaagaggttcactattccctagcatgttgcgaaaaactcattgagttaaatttgtgtttgtgtttcaagcttaggagatttccatgggttaataacatgaaatctatgaatctacgatgttgctatagtttaatggaatttccagaattcctcggtacaatgaagccagagttagtgagtctcaaagtaaacagtaggataagggaactaccattatctattcagtacctaattcatctaacagagctagatttgagaaacatggaaacccttgaagctcttccaagcagcattggtaagttgaaaggtttggtgaagctaaatgtgtcgcgctgcttcacaattaaaagcttgcccgaagagataggtgatttagaaaacttggaggaacttgatgcttcatatactctaatttcacgacctccgtcttccattgtccgcttgaacaagcttaaatacttgaagtttgtaagaatgaaaacagaagatgaagtgtactttgtgtttcctccaattaatggcgggttactctcattggaaatcctggagctcggttcctccaaattcatagatggaagaattccagaagatattggatacttatcctctttgaaagatttgcatctcgagggagataattttgagcatttgcctcaaagcatagcccaacttggtgctcttcggttcttgcacttagtacgttgcacgaggcttacacagctgccagaatttccaccacaattagatacaatatatgcagattggcgcaatgatttgatctgtaattcactgtttcaaaatatctcatcattccagcatgacatctctgcttcagattccttgtcgttaagagtgtttacgagttcggggagtaatataccaagttggttccactatcagggaatggataaaagtgtttcagtcaatttgcctgaaaactggtatgtatcagataacttcttgggatttgctgtatgttactctggaagtttaattgaaaactcggctcaattgattattagttctgaagggatgccgtgtatcacccagaaacttgtcttatccaatcattcagaatataaatatttcaagtttcggtttttcttggtaccttttgctggcatatgggatacatctaacgcaaatggtaaaacaccgaatgactatgggcacattatgttatcttttcctgaagaattggagggatgtggacttcgtttgttctataaagatgaatctgagcttgttgagaccaatgatgaaccatcaacagaactttcccttgggataaggaggatcagatacgacgatagtgaacatcatgaagaagccagttgttcatcttctaagaaacaaaggtcataa

SEQ ID NO: 4 - protein, 1128 aa, Solanum sparsipilum; identical to SEQ ID NO: 2

SEQ ID NO: 5 - DNA, 2938 bp, Solanum sparsipilum
  5′UTR (1)..(164)
  CDS (165)..(2585)
  3′UTR (2586)..(2938)
aaatatcaggttctagggcatttccacatttgtaaagtgaatggacaacagccatatcctccgccgccttgtctgaattgtcaatacaaggactttgtccaaaatatcatcaattgatctcatccataaaaaatatttttcttttcagacaattaatcgaatctatggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggattgaaatgcatgatttgattgaagatatgggtagatatgtggtgaaaatgcaaaagtttccgaaaaaacgtagcagaatatgggatgctgaagatttcaaaaaagtgatgatagactatacagggaccatgacagtggaagcaatctggtttaggtgctataaagaagaactatactttaataatgaggcaatggaaaaaatgaaaagtcttaggatattacaagttgatggttataacaaattctttgcttcacgtccctcttcgaatcaccatgatgactctattgagcacatgtccaataacttgcgttggttagtctggaatcactattcttggaagtcattgccagaatattttaaaccagaaaagcttgtacatcttgaacttcgtaacagttcgctgcattatttatggaaggaaacaaagcatttgccgtctctacgaaagctagatctcagctattctgaaagcctggtgcaaacaccagatttcacggggatgccaaatttggagtatttgactctggagggctgtagaaagcttgaagaggttcactattccctagcatgttgcgaaaaactcattgagttaaatttgtgtttgtgtttcaagcttaggagatttccatgggttaataacatgaaatctatgaatctacgatgttgctatagtttaatggaatttccagaattcctcggtacaatgaagccagagttagtgagtctcaaagtaaacagtaggataagggaactaccattatctattcagtacctaattcatctaacagagctagatttgagaaacatggaaacccttgaagctcttccaagcagcattggtaagttgaaaggtttggtgaagctaaatgtgtcgcgctgcttcacaattaaaagcttgcccgaagagataggtgatttagaaaacttggaggaacttgatgcttcatatactctaatttcacgacctccgtcttccattgtccgcttgaacaagcttaaatacttgaagtttgtaagaatgaaaacagaagatgaagtgtactttgtgtttcctccaattaatggcgggttactctcattggaaatcctggagctcggttcctccaaattcatagatggaagaattccagaagatattggatacttatcctctttgaaagatttgcatctcgagggagataattttgagcatttgcctcaaagcatagcccaacttggtgctcttcggttcttgcacttagtacgttgcacgaggcttacacagctgccagaatttccaccacaattagatacaatatatgcagattggcgcaatgatttgatctgtaattcactgtttcaaaatatctcatcattccagcatgacatctctgcttcagattccttgtcgttaagagtgtttacgagttcggggagtaatataccaagttggttccactatcagggaatggataaaagtgtttcagtcaatttgcctgaaaactggtatgtatcagataacttcttgggatttgctgtatgttactctggaagtttaattgaaaactcggctcaattgattattagttctgaagggatgccgtgtatcacccagaaacttgtcttatccaatcattcagaatataaatatttcaagtttcggtttttcttggtaccttttgctggcatatgggatacatctaacgcaaatggtaaaacaccgaatgactatgggcacattatgttatcttttcctgaagaattggagggatgtggacttcgtttgttctataaagatgaatctgagcttgttgagaccaatgatgaaccatcaacagaactttcccttgggataaggaggatcagatacgacgatagtgaacatcatgaagaagccagttgttcatcttctaagaaacaaaggtcataagtagagggaggttggaaaaacgatgaagcattaatctaagtgtgagacagtggaggaagtagtttgtgcatacactgcggatgatcaatctagcacgtggattaagtagctcagatcgagcaattacattgttttttcatttagtgtctggtacctgtcatagggtttccacttaatgtaattcaaatgccacactgttggaaccaattgacttaagccaattaaaaattattttgtcaatcccttgttaccgatcaaatgttgtgctttaggaagttgtgaaaagggaaaatgaacaaatgatcccttgtgatttttttttatttgaataaacataatgaaagtttagccaa

SEQ ID NO: 6 - protein, 806 aa, Solanum sparsipilum
MASSSSSASNLKYCPRWKYVVFLSFRGEDTRKTFTGHLYEGLKNRGISTFQDDKRLEHGDSIPKELLRAIEESQVALIVFSKNYATSRWCLNELVKIMECKDEENGQTVIPIFYDVDPSHVRNQSESFGAAFAEHELKYKDDVEGMQKVQRWRNALTVAANLKGYDIRDRIEMHDLIEDMGRYVVKMQKFPKKRSRIWDAEDFKKVMIDYTGTMTVEAIWFRCYKEELYFNNEAMEKMKSLRILQVDGYNKFFASRPSSNHHDDSIEHMSNNLRWLVWNHYSWKSLPEYFKPEKLVHLELRNSSLHYLWKETKHLPSLRKLDLSYSESLVQTPDFTGMPNLEYLTLEGCRKLEEVHYSLACCEKLIELNLCLCFKLRRFPWVNNMKSMNLRCCYSLMEFPEFLGTMKPELVSLKVNSRIRELPLSIQYLIHLTELDLRNMETLEALPSSIGKLKGLVKLNVSRCFTIKSLPEEIGDLENLEELDASYTLISRPPSSIVRLNKLKYLKFVRMKTEDEVYFVFPPINGGLLSLEILELGSSKFIDGRIPEDIGYLSSLKDLHLEGDNFEHLPQSIAQLGALRFLHLVRCTRLTQLPEFPPQLDTIYADWRNDLICNSLFQNISSFQHDISASDSLSLRVFTSSGSNIPSWFHYQGMDKSVSVNLPENWYVSDNFLGFAVCYSGSLIENSAQLIISSEGMPCITQKLVLSNHSEYKYFKFRFFLVPFAGIWDTSNANGKTPNDYGHIMLSFPEELEGCGLRLFYKDESELVETNDEPSTELSLGIRRIRYDDSEHHEEASCSSSKKQRS

SEQ ID NO: 7 - DNA, 1638 bp, Solanum sparsipilum
  CDS (1)..(1638)
atggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggattgaatcagagcatattcaacagatcgtagactacatctattccaaattttgcacaaatgcttattcttcatcttttttgcaaggtgttgtgggaataaacgatcacttagagaaactaaaatccaaacttcaaatggaaatcaacgatgttcggattttagggatatggggaataggcggagtcggtaaaacgacaatagcaaatgccatttttgatactatatcttatcaatttaaagcttcctgttttcttgcagatgttaaagaaaatgcaaaaaagaatgaactgtattctttacaaaatacccttctctctgaactgttaagaaaaaaagatgattatgtcaataataagtatgctgggaagcgcatgattccgagcatactttggtctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaggctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaacaactgtagagcaaataaagaaaaactctaattcagaaattgttgaaaaactcaaaataagttatgatgggttggagctcgaagagcaagagatatttctagatattgcatgtttcttacgtgaaattgaaagaaaagaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagtttccgaaaaaacgtagcagaatatgggatgctgaagatttcaaaaaagtgatgatagactatacagtaagtaagctaaacaatgcaataatgtttaatttctaa

SEQ ID NO: 8 - protein, 545 aa, Solanum sparsipilum
MASSSSSASNLKYCPRWKYVVFLSFRGEDTRKTFTGHLYEGLKNRGISTFQDDKRLEHGDSIPKELLRAIEESQVALIVFSKNYATSRWCLNELVKIMECKDEENGQTVIPIFYDVDPSHVRNQSESFGAAFAEHELKYKDDVEGMQKVQRWRNALTVAANLKGYDIRDRIESEHIQQIVDYIYSKFCTNAYSSSFLQGVVGINDHLEKLKSKLQMEINDVRILGIWGIGGVGKTTIANAIFDTISYQFKASCFLADVKENAKKNELYSLQNTLLSELLRKKDDYVNNKYAGKRMIPSILWSMKVLIVLDDIDHSEHLEYLAGDVDWFGNGSRVIVTTRNKHLIEKDDAIYEVSTLPDHEAMQLFNKHAFKKEDPDESFKKFSLEVVNHAKGLPLALKVWGSLLHKKCLTLWRTTVEQIKKNSNSEIVEKLKISYDGLELEEQEIFLDIACFLREIERKEVMQILESCDFGAEYGLNVLINKSLVFISENDRIEMHDLIEDMGRYVVKMQKFPKKRSRIWDAEDFKKVMIDYTVSKLNNAIMFNF

SEQ ID NO: 9 - DNA, 1992 bp, Solanum sparsipilum
  CDS (1)..(1992)
atggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggattgaatcagagcatattcaacagatcgtagactacatctattccaaattttgcacaaatgcttattcttcatcttttttgcaaggtgttgtgggaataaacgatcacttagagaaactaaaatccaaacttcaaatggaaatcaacgatgttcggattttagggatatggggaataggcggagtcggtaaaacgacaatagcaaatgccatttttgatactatatcttatcaatttaaagcttcctgttttcttgcagatgttaaagaaaatgcaaaaaagaatgaactgtattctttacaaaatacccttctctctgaactgttaagaaaaaaagatgattatgtcaataataagtatgctgggaagcgcatgattccgagcatactttggtctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaggctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaacaactgtagagcaaataaagaaaaactctaattcagaaattgttgaaaaactcaaaataagttatgatgggttggagctcgaagagcaagagatatttctagatattgcatgtttcttacgtgaaattgaaagaaaagaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagtttccgaaaaaacgtagcagaatatgggatgctgaagatttcaaaaaagtgatgatagactatacagggaccatgacagtggaagcaatctggtttaggtgctataaagaagaactatactttaataatgaggcaatggaaaaaatgaaaagtcttaggatattacaagttgatggttataacaaattctttgcttcacgtccctcttcgaatcaccatgatgactctattgagcacatgtccaataacttgcgttggttagtctggaatcactattcttggaagtcattgccagaatattttaaaccagaaaagcttgtacatcttgaacttcgtaacagttcgctgcattatttatggaaggaaacaaaggtaccattttatttaagctactttctaaggaaaagggtaaatacccctcaactttgtgatttgaaactgatatatcctttgttttaa

SEQ ID NO: 10 - protein, 663 aa, Solanum sparsipilum
MASSSSSASNLKYCPRWKYVVFLSFRGEDTRKTFTGHLYEGLKNRGISTFQDDKRLEHGDSIPKELLRAIEESQVALIVFSKNYATSRWCLNELVKIMECKDEENGQTVIPIFYDVDPSHVRNQSESFGAAFAEHELKYKDDVEGMQKVQRWRNALTVAANLKGYDIRDRIESEHIQQIVDYIYSKFCTNAYSSSFLQGVVGINDHLEKLKSKLQMEINDVRILGIWGIGGVGKTTIANAIFDTISYQFKASCFLADVKENAKKNELYSLQNTLLSELLRKKDDYVNNKYAGKRMIPSILWSMKVLIVLDDIDHSEHLEYLAGDVDWFGNGSRVIVTTRNKHLIEKDDAIYEVSTLPDHEAMQLFNKHAFKKEDPDESFKKFSLEVVNHAKGLPLALKVWGSLLHKKCLTLWRTTVEQIKKNSNSEIVEKLKISYDGLELEEQEIFLDIACFLREIERKEVMQILESCDFGAEYGLNVLINKSLVFISENDRIEMHDLIEDMGRYVVKMQKFPKKRSRIWDAEDFKKVMIDYTGTMTVEAIWFRCYKEELYFNNEAMEKMKSLRILQVDGYNKFFASRPSSNHHDDSIEHMSNNLRWLVWNHYSWKSLPEYFKPEKLVHLELRNSSLHYLWKETKVPFYLSYFLRKRVNTPQLCDLKLIYPLF

SEQ ID NO: 11 - DNA, 3793 bp, Solanum sparsipilum
  5′UTR (1)..(164)
  CDS (165)..(692)
  3′UTR (693)..(3793)
aaatatcaggttctagggcatttccacatttgtaaagtgaatggacaacagccatatcctccgccgccttgtctgaattgtcaatacaaggactttgtccaaaatatcatcaattgatctcatccataaaaaatatttttcttttcagacaattaatcgaatctatggcatcttcttcttcttctgcgagtaatttaaagtattgtcctcgatggaagtacgttgtgtttctaagtttcagaggtgaagacactcgaaaaacatttacgggtcacttgtatgaaggtttgaaaaataggggaataagcacttttcaagatgataagaggctagagcatggagattcaattccgaaagaactcttgagagctatcgaagagtctcaagttgcacttattgttttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcagaaggtgcaaagatggagaaatgctctaactgttgccgcaaatctaaaaggatatgatatccgtgaccggttagttgaatacacataattacttttaatgaaaaaatggaatcatttccattcaaacacaatataatttcattgattattatttagatggtagagtatatacatttttattgttaagaaggcatagttctatcaatttaattagagaggatacataaaagtcctcctaaactatcaatcttcttctttatgtaggattgaatcagagcatattcaacagatcgtagactacatctattccaaattttgcacaaatgcttattcttcatcttttttgcaaggtgttgtgggaataaacgatcacttagagaaactaaaatccaaacttcaaatggaaatcaacgatgttcggattttagggatatggggaataggcggagtcggtaaaacgacaatagcaaatgccatttttgatactatatcttatcaatttaaagcttcctgttttcttgcagatgttaaagaaaatgcaaaaaagaatgaactgtattctttacaaaatacccttctctctgaactgttaagaaaaaaagatgattatgtcaataataagtatgctgggaagcgcatgattccgagcatactttggtctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaggctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaacaactgtagagcaaataaagaaaaactctaattcagaaattgttgaaaaactcaaaataagttatgatgggttggagctcgaagagcaagagatatttctagatattgcatgtttcttacgtgaaattgaaagaaaagaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagtttccgaaaaaacgtagcagaatatgggatgctgaagatttcaaaaaagtgatgatagactatacacatttgccgtctctacgaaagctagatctcagctattctgaaagcctggtgcaaacaccagatttcacggggatgccaaatttggagtatttgactctggagggctgtagaaagcttgaagaggttcactattccctagcatgttgcgaaaaactcattgagttaaatttgtgtttgtgtttcaagcttaggagatttccatgggttaataacatgaaatctatgaatctacgatgttgctatagtttaatggaatttccagaattcctcggtacaatgaagccagagttagtgagtctcaaagtaaacagtaggataagggaactaccattatctattcagtacctaattcatctaacagagctagatttgagaaacatggaaacccttgaagctcttccaagcagcattggtaagttgaaaggtttggtgaagctaaatgtgtcgcgctgcttcacaattaaaagcttgcccgaagagataggtgatttagaaaacttggaggaacttgatgcttcatatactctaatttcacgacctccgtcttccattgtccgcttgaacaagcttaaatacttgaagtttgtaagaatgaaaacagaagatgaagtgtactttgtgtttcctccaattaatggcgggttactctcattggaaatcctggagctcggttcctccaaattcatagatggaagaattccagaagatattggatacttatcctctttgaaagatttgcatctcgagggagataattttgagcatttgcctcaaagcatagcccaacttggtgctcttcggttcttgcacttagtacgttgcacgaggcttacacagctgccagaatttccaccacaattagatacaatatatgcagattggcgcaatgatttgatctgtaattcactgtttcaaaatatctcatcattccagcatgacatctctgcttcagattccttgtcgttaagagtgtttacgagttcggggagtaatataccaagttggttccactatcagggaatggataaaagtgtttcagtcaatttgcctgaaaactggtatgtatcagataacttcttgggatttgctgtatgttactctggaagtttaattgaaaactcggctcaattgattattagttctgaagggatgccgtgtatcacccagaaacttgtcttatccaatcattcagaatataaatatttcaagtttcggtttttcttggtaccttttgctggcatatgggatacatctaacgcaaatggtaaaacaccgaatgactatgggcacattatgttatcttttcctgaagaattggagggatgtggacttcgtttgttctataaagatgaatctgagcttgttgagaccaatgatgaaccatcaacagaactttcccttgggataaggaggatcagatacgacgatagtgaacatcatgaagaagccagttgttcatcttctaagaaacaaaggtcataagtagagggaggttggaaaaacgatgaagcattaatctaagtgtgagacagtggaggaagtagtttgtgcatacactgcggatgatcaatctagcacgtggattaagtagctcagatcgagcaattacattgttttttcatttagtgtctggtacctgtcatagggtttccacttaatgtaattcaaatgccacactgttggaaccaattgacttaagccaattaaaaattattttgtcaatcccttgttaccgatcaaatgttgtgctttaggaagttgtgaaaagggaaaatgaacaaatgatcccttgtgatttttttttatttgaataaacataatgaaagtttagccaa

SEQ ID NO: 12 - protein, 175 aa, Solanum sparsipilum
MASSSSSASNLKYCPRWKYVVFLSFRGEDTRKTFTGHLYEGLKNRGISTFQDDKRLEHGDSIPKELLRAIEESQVALIVFSKNYATSRWCLNELVKIMECKDEENGQTVIPIFYDVDPSHVRNQSESFGAAFAEHELKYKDDVEGMQKVQRWRNALTVAANLKGYDIRDRLVEYT

SEQ ID NO: 13 - DNA, 23 bp, Artificial
  Amorce MS063_2F
tcacccaaaaagtaaattcaaaa

SEQ ID NO: 14 - DNA, 20 bp, Artificial
  Amorce MS063_2R
cgcctcgtgtcacaataaga

SEQ ID NO: 15 - DNA, 22 bp, Artificial
  Amorce MS092_F
aggtcatcatcacacacaacaa

SEQ ID NO: 16 - DNA, 25 bp, Artificial
  MS092_R
gggatgtgaaataacaaacctttac

SEQ ID NO: 17 - DNA, 20 bp, Artificial
  Amorce ASC102_F
ggcaaacgagagagtgacga

SEQ ID NO: 18 - DNA, 27 bp, Artificial
  Amorce ASC102_R
gagtcgatttttgtatatgtttgagaa

SEQ ID NO: 19 - DNA, 29 bp, Artificial
  Amorce ASC231_F
tttatatgatattatttaacgtgacaagg

SEQ ID NO: 20 - DNA, 22 bp, Artificial
  ASC231_R
tttgtcttaggtcatgatggtg

SEQ ID NO: 21 - DNA, 25 bp, Artificial
  Amorce ASC240_F
tcatatagagcaccaaagtttttcc

SEQ ID NO: 22 - DNA, 21 bp, Artificial
  Amorce ASC240_R
tgtttgttcccattctgatga

SEQ ID NO: 23 - DNA, 20 bp, Artificial
  Amorce AZ751_2F
ttgggatttgctttgttggt

SEQ ID NO: 24 - DNA, 23 bp, Artificial
  Amorce Z751_2R
catgagaattcagatagtcaacg

SEQ ID NO: 25 - DNA, 30 bp, Artificial
  Z1505_6F
tcaatcccacaaaagtccttcaactaaacc

SEQ ID NO: 26 - DNA, 31 bp, Artificial
  Amorce Z1505_R
acacccaattagctttaactagataccaagg

SEQ ID NO: 27 - DNA, 29 bp, Artificial
  Z1505_8F
gtggagcatgggttttcgttatctttatc

SEQ ID NO: 28 - DNA, 31 bp, Artificial
  Amorce Z1505_4R
attcactcccttaaagcccataactcaacag

SEQ ID NO: 29 - DNA, 21 bp, Artificial
  Amorce Q63F
aacttctcgcttagctttctg

SEQ ID NO: 30 - DNA, 18 bp, Artificial
  Q63R
tccctgccataacatgag

SEQ ID NO: 31 - DNA, 31 bp, Artificial
  Amorce Z1505_5R
gtaaagaattcactcccttaaagcccataac

SEQ ID NO: 32 - DNA, 20 bp, Artificial
  Amorce Z3461F
gaccatgacagtggaagcaa

SEQ ID NO: 33 - DNA, 22 bp, Artificial
  Amorce Z3461 R
tggttgtcagaagcaaatgaag

SEQ ID NO: 34 - DNA, 10030 bp, Solanum sparsipilum
gtggagcatgggttttcgttatctttatcggagatgttgaaggttagtgatggggaattcaatgccagattgattgaaatgcgattgaggatagtggaagataagcaattgtagtaagtaagcaattcttttctatgagtagagtcgatttttgtatatgttggagaatcattagagttaatactaaatgttttaacatatattcattgtggttagatgcacatttttgtgataaagtaatgattttatccttgatgtggtcttgttcatggtttttattgtttctcgtggaatgcaatttgaagtttgctatgctcgtgttgtgttgtttaagtactaatttttatccatgatgtggtattctctgtacatggtttttattgtttcatgtggaacgcgattcaaatttgatatgctcttgttttttataacggatatcatccgcagatgatttatcgtgttgaagtattgctctcgaagatgcatgttcatctagtgacttgcttctcaaagattgcatcttttttgttctgtactttgtatagttgcgaccagatataattatagctacgcctatggtagaggggtgtgatttttcttggtgtggtaggagattttaggccttaaaacaggcattgctttgtgtatacaataataatgagactcactgaccactgctctgtccttactattgaagttaggattcatctttttagatatccgaggctcttaacaaattaagatgttaatttgattctatactatattaaaaatagtgaaagaaaatattacaatactggttgaaaagttagttgaatgaagaaaccatcagatcaaagttcaaaatttgagtagttgatgttcttgaagttgtgaaagtattatcgaaatagattcaatccaaatatttggggatgacccaaaagggaagtgtcatacaaaattggaatggatacgatactattttagtgtgcttgcaactttgcaagggttagttcattgtcattggcgatgatgcataagtttaatggttgaatgactatgttatacgttagaataattgaagtaagatagaaacttgttatgcgagaattctgttgcattctcttttctcatgctgaaacaatggttcaacaacgggtagtaagcaattaataacatatgtccatgctttaactgttcctttctataacacctcgtttgacattgctcagctttctgccggccagaaatttgtgcaactcttacatttctataagtagctcgatcccagactggttctactgaaagatgtgcttctccacgtctgtagaaattcgttagactttatggtctaatttgttgttagacatattcttgttggtatttcgttatctggctctatagttactcgaaatcttatgtctagggcattcttctataatatcaaacatgtactttcgaagcttgaaggttgctattgtagtttcatccttgttgttgaaccttgggaagagttgttatctaccagtcatgtgcttctgatatttgagagttaaagttggggtagtgttttgccctctgttaaacctcaaattatgttttgttccaattgaacacataatgaagtcttctcataattgattttatggaatactacattttattagaacaagctttgaggagctatattacatactcaaatatatatactgcatatggttttttttgtgatgcaaataacaccattaatattgatcgtcactctctcgtttgccaaaaaaaatatttataccatacatgtatatggtaatttatgcataaattttataaaatgacaagtattgtgatccagctatttatagaaagggatgacatgtaaaatgggacagagggagtattttttattattaatatgtaaacaataaaccagaaaataaggaggaaaagaaaggaaaaaaaacatttatagactataaatgaaggagaatatgaatgtcatttaactcttattaaattgattgaattaagcgtgaacattaaagtactattgcttcatttcaaatcatccgtttaattatgattttataatattgagggtaaaagtaaatattcataaactactccctccattccatattaatttaacttttgagatattcttcatttttcaaattcggacctttgttttttaatcaaatttttgggcctgctggcctttttatttaactttctgaaactaacattcagtacaaggactttggcccaaataaaaaataccaaaacaaaataaaattaatctagaagatccaatcatccaacccaacaaatatcaggttctagggcatttccacatttgtaaagtgaatggacaacagccatatcctccgccgccttgtctgaattgtcaatagactttgtccaaaatatcatcaattgatctcatccataaaaaaaaaatttcttttcagacaattaatcgaatctatggcatcttcttcttcttctgcgagtaattcaaagtattgtcctccatggaagtacgttgtgtttctaagtttcagaggtgaagacactcgaaaaacatttacgggtcatttgtatgaaggtttgaaaaataggggaataagcacttttcaagatgataaaaggctaaagaatggagattcaattccgaaagaactcttgagagctatcgaagagtctcaagttgcacttattgttttctcaaagaattatgctacatctaagtggtgcttgaatgaactagtgaagatcatggaatgcaaagatgaagaaaatggacaaacagtcataccaatcttctatgatgtggatccatcacatgttcgaaaccaaactgaaagctttggagcagcatttgccgaacatgaattaaagtataaggatgatgttgaggggatgcagaaggtgcaaaaatggagaaatgccctaactgttgccgcaaatctaaaaggatatgatatccgtgacgggttagttgaatacacataattacttttaatgaaaaaatggaataatttccattcaaacacaatataatttcattgattattaattatttagatggtagagtatatacatttttattgtttagaaggcatagttctattaatttaattagagaggatacatcaaagtcctcctaaactatcaatcttcttctttattctttatgtaggattgaatcagagaatattcaacagatcgtagactgcatctcttccaaattttgcacaaatgcttattcttcatcttttttgcaagatgttgtgggtataaatgatcacttagagaaactaaaatccaaacttcaaatggaaatcaacgatgttcagattttagggatctggggaataggcggagtcggtaaaacgacaattgcaaaagccatttttgatactatatcttatcaatttaaagcttcctgttttcttgcagatgttaaagaaaatgcaaaaaagaatgaactgcattctttacaaaatacccttctctctgaactgttgagaaaaaaaaatgattacgtcaataataagtatgatgggaagcgcatgattccgagcatactttgttctatgaaggtgctaattgtgcttgatgatatagatcacagtgagcatttggagtatttagcaggtgatgttggttggtttggtaatggcagtagagtcattataacaactagaaacaaacatttgatagagaagcatgatgcaatatacgaagtgtctacactacctgatcatgaagctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaataactgtagagcaaataaagaaaaactctaattcagaaattgttgaaaaactcaaaataagttatgatgggttggagtccgaagagcaagagatatttctagatatcgcatgtttcttccgtggaaatgaaagaaaaaaagtcatgcaaattcttgagagttgcgactttggagctgagtacggattgaatgttctgattaataaatctcttgtgttcatctctgaaaataataggattgaaatgcatgatttgattgaagatatgggtagatatgtggtgaaaatgcaaaagcttccgaaaaaacgtagcagaatatgggatgttgaagatgtcaaaaaagtgatgatagactatacagtaagtaagctaaacaatgcaataatatttaatttctaatttttatatttcaaagacgtataagccaatcaattccaattatttgttcctcttgcttcatattcttacaggtacgtcattttagctctttactttactttatttatttattttttaataaaagaagcaaaagtaacatcaattgcctaacatagttaatcgtctatgaattagtgtttactatgttttatccagtttctttcttaagattgcatgttatacacaccaataagtaaatagtttttaaagctaatatcacccacatatttaccctataaaaatcctcacataaaaaaatacaagtaagaagggtcaaatcataacataccaatcctcaaccaagaattcacttaatcattgatttttaaaaaaaaataattaaaagatgacaattatttgtattgttccaattacaatatttattctatcactgcattgaattttttatgatttgatgctaaagaacttctaaaaaaatagtattattagaaaatataaatatttgcacaaaatatttcttgtaagcaacatgtgaagttatgattagttgactcatgatctttttatcaggggaccatgacagtggaagcaatctggtttacgtgctatgaagaagaactatgctttaataatgaggcaatggaaaaaatgaaaagtcttaggatattacaagttgatggtgttatcaaattctttgcttcacgtccctcttcgaatcaccatgatgactctattgagtacctgtccaataacttgcgttggttagtctggtatcactattcttggaagtcattgccagaaaactttaaaccagaaaagcttgttcatctcgacctttattgcagttcgctgcattatttatggaaggaaacaaaggtaccattttatttaagctactttctaaggaaaagggtaaatacccctcaactttgcaatttgtaactgatatatcctttgtttaaaaaaatgatatatatactatgtcgtctaataaatggtgcatatgtaccatttccattaacgggtctgtatttattgaattaaaaaaataattataaaattgatttttaaaattcaaaaatatcatgtggctttaaaaattaccccactcgtttttttgcccctctaaacccaacccaaatgaataaaaacccaatccatcttctactcaaacataagatgagtttgagtcaggtctgaataaagagggatgaggttctttagttgggttaggtctagatgggttgtgtgagtaatttaaaaaaaaaaccatgtgagtgtttagaaatttaaaaactaacttcaaattttttaaaatttaattgttaatgaaaataataaatatgcattatttgttaaacgttcatgttatatatgcatcattttattttttttaacaaaaggtatattcactccagatcacatagtttagatgtatatctgccccttttttctactttctatggttacttgtcctagaattatggtgtttgtatttaaatgaaataagttattaattccataaactcaaattaatatatgttatacaatattgagttttcatattgttactatgaaaaagggctaaaattgtccttaaactaacaattatagctcaattaatccttcatttgcttctgacaaccaaaacacccggccatctattttctgatggatttaatgtaacctacacttttaaacaatcaagttaagttcgagtttaaaaaaatagattaggtaagaatcactcattgatacgacgaaacccaacgtcatataaatgtaaaaattaactaagcaatttcttcaaaggcaatttaaccctattttatcatattgaattgtcatgtcagctttttctattatttaaatggccctaacatgctcatgtggacaacattagcttggaggcatttttttgtccaaaaaatagatagcaagggtatttttggtataccaaaagcaaatgaaggataaaatggagctatttctgatagtttaagggcaatttagtcccttttctattaaaattataatatcgttatgtcttttttttcaaagcttgcctcattattttaactaagaagctcaatttaaaatttgatcattcaaatatttacctacaaaaatatgaatctcatcctaggttttatgtagtgggataaaatgaatacatatcgaagtagttcattctatactactatcttcttgaatgatgattaaggtttcatatattggttttgggctaaaactataacatatctataggaataggttcattttggtcttcaaatatatataatatgaacatttttaatcccttcattttgctaaagtggagtacttttaatctccttaataaaatccgtttaaaagtaacagtgttaaactcaagtaacattcacatgactcttaaaacataaaatccattgggtaaattcattccccgtttaatagttgtctagatgccatattcttaatatttgatttacaccgacaactaaacagtctaaacactctcaccaaaagaaaaaaaaagataattaaaataaataatcattacagctaaagaattaaaaaaaaaaaaacataaattggggttaaaattatgccctctatttttcttttcactcaagcacctagaagagtgtattcactcttttccacatcaatgttcaagagataaatgatattccctccgtttcacaaagaatgacttacctttttttttagtcagtttaaaaaagaatgacctttttctatttttggtaacattttaatttcagttttccacgtgacatgtttaaggccacaagattaaaagacaattttgtacatttgacataactttaatgtaggaccacagattcaaaagtcttctttattttcttaaacgtcgtgtcaagtcaaactagaccattctttgtgaaattgagagagtagtaaatagtcaagtatatataggggatatttaagataagtgtaacaatcaagtgtgtattgaataaaacatgtcaagtctaaaggattatagataataaacacttttatttagtttctgaacatgtaatttccatatcaaaataaaataaaacattttctaatttcacttaaaatattgtgttagactatcataatccaaatgtctttattcatttttggtctaaggggtaaattaaacacaaagatcattctttctctattttgaatagcagcatttgccgtctctacgaaagctagatctcagcttttctaaaagcctggttcaaacaccagatttcacggggatgccaaatttggagtatttgaatctggatggctgtagtaagcttgaagaagttcactattccctagcatattgcgaaaaactcattgagttaaatttgaattggtgtaccaatcttaggagatttccatgggctaacatgaaatctcttcaaactatgaatctacaggcttgctttagtttaatggaatttccagaattcctcggtacaatgaagccggagttagcgagtctcgcagtaaacagtgggataagggaactaccatcatctattcagtacccaactcatctaacaaagctagatttgagtggcatgggaaaccttgaagctcttccaagcagcattgttaagttgaaaggtttggtgaagcttaatgtgtcgtactgcataacaattaaaagcttgcccgaagagataggtgatttagaaaacttggaggaacttgatgctacaggtactctaatttcacgacctccgtcttccattgtccgcttgaacaagcttaaatccttgaagtttgcaaaagaaaaaacagaagatgaagtgtactttgtgtttcctccaattaatggcgggttactctcattggaaatcctggagctcggttcctccaaattcatagatggaagaattccggaagatattggatacttatcctctttgaaaaagttgcatctccaaggagataattttgagcatttgcctcaaagcatagcccaactcggtgctcttcggttcttgtacttagtatattgcacgaggcttactcagctgccagaatttccaccacaattagatacaatatgtgcagattggcgcaatgatttgatttgtaattcactgtttcaaaatatctcatcattccagcatgacatctctgcttcagattccttgtcgttaagagtgtttacgagtgagtggagttatatccctagttggttccactatcagggaatgggtaaaagtgtttcagtcaatttgcctgaacactggtatgtatcagataacttcttgggatttgctgtatgttactctggaagtttaattaaagacacggctcagttgattattagttctgaagggatgccgtgtatcacccagaaacttgtcttatccaacaattcagaaggtcgtccatattctacgtttctgtttttctttgtaccttttgctggcatatgggatacatctaacgcaaatggtaaaacaccaaatgactatgggcacattatgttatcttttcctgaagaatggatggagtgtggacttcgtttgctctataaagatgaatctgagcttgttgagaccagtgatgaaccatcaacagaactttccattgagaccagtgatgaaccatcaacagaactttccattgggataaggaggatcagatacgacgatagtgaacatcatgaagaagccagttgttcctcttctaagaaacaatggtcataagtagaggtatatatctcgatatgtatctaaacttctcgctttgctttctgttattaatagttcattttgatgctctttgtggttattataatagaggaaatttttgttctaaatacaaatatctctcatgttatggcagggaggttggaaaaacgaggtgaagttgaggttcgtcctccctcatgattgatctcttttggaccagattacctagtttcactgtccaaaagaagtgtggataatctacatggtagaagtagtttgtgcatacactgcggatgatcaatctagcacgtggattaagtagttcagatcgagcaactacattgttttttcatccagtgtttggtacctgtcatagggtttccacttaatgtaattcaaatgtcacattgttggaaccaattgacttaagccaattaaaaatatttttgtcaatcccttgttaccgaacaaatgttgtgctttaggaagttgtgaaaagggaaaatgaacaaatgatcccttgtgatttttttttatttgaataaacataatgaaagtttagccaattgacaagtttaagaggttttagttattttaccttttatttataataataataatacttttatttagtttcttgggtgtgcaatagtttgagaaacggaagatggtactttgacaatatcgatgacactaagatttcgcgaaagaacttgggtatgcaatagtttaaagttttattttgaaaatataatcacattttccatttgcttaaaagtaactggtccaaaaaaaatcaaaagaacttgggtatgcaatagtttatgggccttttgaagtagtatcaacgttgctggcctcttgggctttaaggcccaagaaggtgggctttctgctaggaaatgaagtagttaaagctaaaagatttgtgtctgtaatatatgtagttaatatatcatgatctcttgagttatgggctttaagggagtgaat

SEQ ID NO: 35 - DNA, 9896 bp, Solanum sparsipilum
gtggagcatgggttttcgttatctttatcggagatgttgaaggttagtgatggggaattcaatgccagattgattgaaatgcgattgaggatagtggaggataagcaaatgtagtaagtaactctttttctatgggtaggatcgatttttgtatatgttggagaatcattagagttaatactaaatgtttaacatatattcattgtggttagatgtacatttttgcgataaagtaatgatttttatccttgatgtggtcttgttcatggtttttattgtttctcgtggaatgcaatacgaagtttgctatgctcgtgttgtgttgtttaagtagtgatttttatccatgatgtggtgttctctatacatggtttttattgtttcatgtggaatgcgattcaaattttgatatgctcttgttttttataatggatatcatccacagatgatttatcgtgttgaagtattgctctcgaagatgcatgttcatctagtaacttgtttctcaaagattgcatttttttgttctgtactttgtatagttgcgaccagatgtaattattgccacacctatgtagagggatgtgattttccttggtgtggtaggagatttagagccttaaaacaggcattgctctgtgtataaaataataatgagactcactaccactgctctgtccttactattgaagttaggattcatctctttagatatccgaggctcttaataaataattactccgttctttttatatgactacattagactggttgaaaagttagttgaatgccgaaaccatcagatcaaagttcaaaattttgaatagttgatgagtaaatatttggggatgacccaaaagggaagtgtcatacaaaattggaatggagacagtactattttagtatgcttgcaactttgcaagggttagttcattggcattggcgatgatgcataagtttaatggttgaatgactatgttatacgttcgaataattgaagtatgctagaaacttgttatgcgagagttcattgcacaaatctctggccggcagaaagctgagcaatgccggccagagatttgtgcaactcttagtggtattacatttctataagtagctcgatcccagactggttctactgaaagatgtgcttctccacgtctgtagaaaatcattatactttgttgttgacatattcttgttggtattccattatctgactctatagatactcgaaatcttatgtctagggcattcttctataatatcaaacatgtactttcgaagcttgaaggttgctattgtagtttcctccttgctgttgaaccttgggaagagttgttatctaccagtcatgtgcttctgatatttgagcgttaaagttgaactagtgttttgccctctgttaaacctcaaattatgttttgttccaattgaacacataatgaagtcttctcatagttgattttatggaatactacattgtattagaacaagctttgaggagctatattacatactcaaatatatatactgcatatgttttttttttttttgtgagcaaataacaccattaatattgatcgtcactctctcgtttgccaaaaaaaatatttataccatacatgtatatggtaatttatgcataaattttataaaatgacaagtattgtggtccagctatttataaaaagggatgacatataaaatgggacggagggagtattttttattattaatatgtaaacaataaaccagaaaataaggaggaaaagaaaggaaaaaataaacatttatagactataaatgaaggagaatatgaatgtcatttaactcttaataaattgattgaattaagcgtgagcattaaagtactattgcttcatttcaaatcatccgtaaaattataattttataatattgagggtaaaagtaaatattcataaactactccctccattccatattaatttaacttttgagatatttttcatttttcaaattcggacctttgctttttaatcaaatttttgtggcctgctggcctttttatttaactttctgaaaccaatattcagtacaaggactttggcccaaataaaaaataccaaaacaacataaaattaatctagaagatccagcccaacaaatatcaggttctagggcatttccacatttataaagtgaatggacaacagccatatcctccgccgcctcgtctgaattgtcaatacaaggactttgtccaaaatataatcaattgatctcatccataaaaaatatttttcttttcagacaattaatcgaatctatggcatcttcttcttcttctgcgagtaattcaaagtattgtcctcgatggaagtacgttgtgtttctaagtttcagaggcgaagacactcgtaaaacatttacgggtcacttgtacgaaggtttgaaaaataggggaataagcacttttcaagatgataaaaggctagagcatggagattcaattccgaaagaactcttgagagctatcgaagtgtctcaagttgcacttattgttttctcaaagaattatgctacatctaagtggtgcttgaatgaactagtgaagatcatggaatgcaaagatgaagaaaatggacaaacagtcataccaatcttctatgatgtggatccatcacatgttcgaaaccaaagtgaaagctttggaacagcatttgccgaacatgaattaaagtataaggatgatgttgaggggatgcagaaggtgcaaagatggagaaatgccctaactgttgccgcaaatctaaaaggatatgatatccgtgacgggttagttgaatacacataattacttttaatgaaaaaatggaatcatttccattcaaacacaatataatttcattgattattatttagatggtagagtatatacatttttattgtttagaaggcatagttctatcaatttaattagagaggatacataaaagtcctcctaaactatcaatcttcttctttatgtaggattgaatcagagcatattcaacagatcgtagactgcatctattccaaattttgcacaaatgcttattcttcatcttttttgcaaggtgttgtgggaataaacgatcacttagagaaactaaaatccaaacttcaaatggaaatcaacgatgttcggattttagggatctggggaataggcggagtcggtaaaacgacaatagcaaaagccatttttgatactatatctgaacaatttaaagcttcctgttttcttgcagatgttaaagaaaatgcaaaaaagaatgagctgcattctttacaaaatatccttctctctgaactgttaagaaaaaaagatgattacgtcaataataagtatgatggggagcgcatgattccgaggaaactttgttctatgaaggtgctaattgtgcttgatgatatagatcacagtgagcatttggagtatttagcaggtgatgttgattggtttggtaatggcagtagagtcattgtaacaactagaaacaaacatttgatagagaaggatgatgcgatatacgaagtgtctacactacctgatcatgaagctatgcaattattcaataagcatgcttttaaaaaagaatatccagatgagagttttaagaagctctcattggaggtagtaaatcacgctaaaggccttcctttagccctcaaggtgtggggttgtttattgcataaaaagtgtctaactttgtggagaataactgtagagcaaataaagaaaaactctaattcagaaattgttgaaaaactcaaaataagttatgatggtttggagctcgaagagcaagagatatttctagatatcgcatgtttcttccgtggaaatgaaagaaaaaaagtcatgcaaattcttgagagttgtgactttggagctgaatacggattgaatgttctgattaataaatctcttgtgttcatctctgaaaatgataggattgaaatgcatgatttgattgaagatatgggtagatatgtggtgaaaatgcaaaagcttccgaaaaaacgtagcagaatatggaatgttgaagatttcaaaaaagtgatgatagactatacagtaagtaagctaaacaatgcaataatatttaatttctaatttttatatttcaaagacgtataagccaatcaattccaattatttgttcctcttgcttcatattcttacaggtacgtcattttagctctttactttatttatttatttattttttaataaaagaagcaaaagtaacatcaattgcctaacatagctaatcgtctatgaattagtgtttaatatgttttatccagtttctttcttaagattgcatgttatacacaccaataagtaaatagtttttaaagctaatatcacccacatatttaccctataaaaatcctcacataaaaaaatacaagtaagaagggtcaaatcataacataccaatcctcaaccaagaattcacttaatcattgattttaaaaaaataaattaaaagatgacaattatttgtattgttccaattacaatatttattctatcactacattgattttttttaaaattttatttgatgctgaagaacttctcaaaaaaaaagtattattagaaaatataaatatttgcacaaaatatttcttgtaagcaacatgtgaagttatgattagttgactcatgatctttttatcaggggaccatgacagtggaagcaatctggtttagctgctatggagaagaaccatgcattaataaagaggcaatggaaaaaatgaaaagtcttaggctattacaagttgatggtttctttgattttcgtctcacttcacgtccctcttcgagtcaccatgatggctctattgagtacctgtccaataacttgcgttggttagtctggaaagagtattcttggaaggcattgccagaaaactttaaaccagaaaagcttgttcatcttgaactctgtttcagttcgctgcattatttatggaaggaaacagaggtaccattttatttaagctactttctaaggaaaagggtaaatacccctcaactttgcgatttggaattgatatatcctttgtttaaaaaaatgatatatatatactctgtcgtctaacaaatggtgcatatgtaccatttccgttaacaaatctgtatttattgaattaaaaataataattataaaattgatttttaaaattcaaaaatatcacgtggctttaaaaaattaccccactcatttttttgcccctttagacccgacccaaatgaataaaaacccaatccatcttctactcaaacataagatgagtttgagtcaggtctgaataaagagggatgaggttctttagttggattaggtctagaggggtaaaaattgagtgagtaatttttttaaaaaccatgtgagtgtttagaaattaaaaaactaacttcaaattttttaaaatttaattgttaatgaaaataataaatatgcattatttgttaaacgttcatgttatatatgcatcattttatttttttaacaaaaggtatattcactccagatcacatagttaagatgtatatttgccccttttttctactttctatggttacttgtcctagaattatggtatttgtatttaaatgaaataagttattaattccataaactcaaattaatatatgttatacaatattgagttttcatattgttactatgaaaaagggataaaattgtccttacactaacaagtatagctcaattaatccttcatttgcttctgacaaccataacaccctgccatctattttctgatggatttaatgtaacatacacttttaaacaatcaagttaagttcgagtttaaaaaaatagattaggtaagaatcactcattgatacaacgaaacccaacgtcatataaatgtaaaaattaaataagcaatttcttcaaaggcaatttaaccctattttgtcatattgaattgtcatgtcagctttttctattatttaaatggccctaacatgctcatgtggacaacattagcttggaggcatattttggtccaaaaattgatagcaagggtatttttggtataccaaaagcaaatgaaggataaaatggagctatttctgatagtttaagggtaatagtcccttttctattaaaattataatatcgttatgtcttttttttcaaagcttgcctcattattttaactaagaagctcaatttaaaatttgatcatgcaaatatttacctacaaaaatatgaatctcatcctaggttttatgtagtgggacaaaatgaatacttatcgaaggagttcattctatactactatcttcttgaatgatgactaaggtttcatatattggatttgggctaaaactataccatatctataggaataggttcattttggtcttcaaatatatataatatgaatattttttatcccttcattttgctaaagtggagtacttttaatctccttgataaaatccatttaaaagtaacagtgttaaactcaagtagcatccacatgactcttattctcttaaaacataaaatccattgggtaaattcattccccgtttaatagttgtctagatgccatattcttaatatttgatctacaccgacaactaaacactctcatcaaaagaaaaaaaaagataattaaaataaatagtcatttcagctaaagaattaaaagaaaacataaattggggttaaaattatgccctctatttttcttttcactcaagcacctagaagagtgtattcacttttttccacatcaatgttcaagaaataaatgatactcccttcgtttcacaaagaatgacctcctttttttttagtcagtttaaaaaagaatgacaattttctatttttggtaacattttaatttcagttttccacgtgacatgtttaaggccacaagattaaaggacaattttgtacatttgacataactttaatgtaggaccacagattcaaaagtcttctttatttcttaaacgtcgtgtcaagtcaaactagtccattctttgtgaaatagagagagtagtaaatagtccagtatatataggggatatttaagataagtgtaacaatcaagtgtgtatagaataaaacatgtcaagtctaaaggattatagatactctaccttttatttagtttctgaacatgtaatttccatataaaaataaaattaaacattttctaatttctctcaaaatattgtgttagactctcataattcagatgtctttattcatttttggtctaaggggtaaattaaacacaaagatcattctttctctattttgaatagcagcatttgccgtctctactaaagctagatctcaccttttctgaaagcctggtgcaaacaccagatttcacggggatgccaaatttggagtatttgaatctgaagcgctgtagtaagcttgaagaggttcactattccctagcatattgcgaaaaactcattgagttaaatttggatagttgttccaagcttaggagatttccatgtgtgaacatgaaatctcttgaatatatgaatctacaagattgctgtagtttaatggaatttccagaattcctcggtacaatgaagccggagttagtgattctcacagcaaactctggaataagggaactaccatcatctattcagtacccaactcatctcacagtgctatgtttgagtggcatgaaaaaccttgaagctcttccaagcagcattgttaagttgaaaggtttggtgaggctagatgtgtcgtgctgcttaacaattaaaagcttgcccgaagagataggtgatttagaaaacttggaggaacttgatgctgcatttactctaatttcacgacctccttcttccattgtccgcttgaacaagcttaaatacttgaagtttgtaaaaaatgaaacagaagatgaagtgtactttgtgtttcctccaattaatggcgggttactctcattggaaatcctggagctcagttcctccaaattcatagatggaaaaattccggaagatattggatacttatcctctttgaaagagttgcgtctccagggagataattttgagcatttgcctcaaagcatagcccaactcggtgctcttcggatcttgtacttagaagattgcaagaggcttacacggctgccagaatttccaccgcaattagataaaatatatgcagattggggcaatgatttgatttgtaattcactgtttcaaaatatctcatcattccaccatgatatctctgcttcagattccttgtcgttaagactgtttacgagtacgtggggttgttgggggagtgatataccacattggttccactatcagggaatggatagaagtgtttcagtcaatttgcctgaaaactggtatgtatcagataacttcttgggatttgctgtatgttactctggaagattaattgaaaacacggctcagttgattattagttctgaagggatgccgtgtatcacccagaaacttgtcttatccaatcattcagaatttaattatttgaacattcagtttttcttggtaccttttgctggcatatgggatacatctaacacaaatggtaaaacaccaaatgactatgggcacattatgttatcttttcctgaagaattggagaagtgtggacttcgtttgttctataaagatgaatctgagcttgttgagaccagtgatgaaccatcaacagaactttccattgagaccagtgatgaaccaccaacagaactttccattgggataaggaggatcagatacgacgatagtgaacatcatgaagaagccagttgttcctcttctaagaaacaaaggtcataagtagaggtatatatctcgatatgtatctaaacttctcgctttgctttctgttattaatagttcattatgatgctctttgtggttattataatagaggaaatttttgttctaaatacaaatatctctcatgttatggcaaggaggttggaaaaaaagagatgaagttgaggttcgccctccctcatgattgatctcttttggagcagattacctagtttcactgtccaaaagaagtgtggataatctacatggtagaagtagtttgtgcatacaatgagcaattacattgttttttcattcagtgtatggtacctgtcataggtttccacttaatgtaattcaaatgccacactgttggaaccaattgacttaagccaattaaaaatatttttgtcaatcccttgttaccgaacaaatgttgtgctttaggaagttgtgaaaagggaaaatgaacaaatgatcccttgtgatttttttttatttgaataaacataatgaaagtttagccaattgacaagtttaagaggttttagttattttaccttttatttataataataataatacttttatttagtttcttgggtgtgcaatagtttgagaaacagttacattttccatttttcatttcgaaaatataatcacattttccatttgctaaaagtaactggtccaaaataaatcaaattgcagatgaatgtatggtaagataattttgcgaaaacgggtggatgaaagaagcctcggaactgtttctagctgaccatccaaatgccttaattgggtgttcatttgagtaaattttaggtttaaacaacataaaaatcatattagagctctatagactgttttgctatttgcgtttaatagcaaattatatgccgtttgtataaaagcgcaatggttgtatatgtatatcagttaaataattgtatatatgtaactactatgtatatgtatcatgatctgttgagttatgggctttaagggagtgaat

SEQ ID NO: 36 - DNA, 10729 bp, Solanum tuberosum
gtggagcatgggttttcgttatctttatcggagatgttgaaggttagtgatggggaattcaatgccagattgattgaaatgcgattgaggatagtggaagataagcaattgtagtaagtaagcaattcttttctatgagtagagtcgatttttgtatatgttggagaatcattagagttaatactaaatgttttaacatatattcattgtggttagatgtacatttttgcgataaagtaatgatttttatccttgatgtggtcttgttcatggtttttattgtttctcgtggaatgcaattcgaagtttgctatgcttgtgttgtgttatttaagtactgatttttatccatgatgtggtgttctctatacatggtttttattgtttcatgtggaacgcgattcaaattctgatatgctcttgttttttataacggatatcatccacggatgatttatcgtgttgaagtattgctctcgaagatgcatgttcatctagtaacttgcttctcaaagattgcatttttttgttctgtactttgtatagttgcgaccagatgtaattatagccacacctatgtagagggatgtgattttccttggtgtggtaggagatttagagccttaaaacaggcattgctctgtgtataaaataataatgagactcactgaccactgctctgtccttactattgaagttaggattcatctctttagatatccgaggctcttaataaatacttactccgttctttttatatgactacattagactggttgaaaagttagttgaatgcagaaaccatcatatcaaagttcaaaattttgaatagttgatgaataactttgtgctgctccacgtctgtagaaaatcgttatactttgttgttgacatattcttgttggtattccgttatctgactctatagatactcgaaatcttatgtctagggcattcttctataatatcaaacatgtactttcgacgaagcttgttgagtgacagttggttgaaccttgaaggttgctattgtagtttcctccttgctgttgagccttgggaagagttgttatctgtcagtcatgtgcttctgatatttgaaagttaaagttggggtagtgttttgccctctgttaaacctcaaattatgttttgttccaattgaacacataataaagtcttctcataaattttatgctaaattgattttatggaatactacattttattagaacaagctttgaggagctatattacatactcaaatatatatatactgcatatgttttttttttgtgatgcaaataacaccattaatattgatcgtcactctctcgtttgccaaaaaaaatatttataccatacatgtatatggtaatttatgcataaattttataaaatgacaagtattgtgatccagctatttatagaaagggatgacatataaaatgggacggagggagtattttttattattaatatgtaaacaataaaccagaaaataaggaggaaaagaaaggaaaaaaaacatttatagactataaatgaaagagaatatgaatgtcatttaactcttattaaattgattgaattaagcgtgagcattaaagtactattgcctcatttcaaatcacccgtttaattatgattttataatattgagggtagaagtaaatattcataaactactccctccattccatattaatttaacttttgagatatttttcatttttcaaattcggacctttgttttttaatcaaatttttgtggcctgctggcctttttatttaactttctgaaaccaacattcagtacaaggactttggcccaaataaaaaataccaaaacaacataaaattaatctagaagatccagcccaacaaatatcaggttctagggcatttccatatttgtaaagtgaatggacaacagccatatcctccgccgccttgtctgaattgtcaatacaaggactttgtccaaaatatcatcaattgatctcatccatttctatggcatcttcttcttcttctgcgagtaattcaaagtattgtcctcgatggaagtacgttgtgtttctaagcttcagaggtgaagacactcgaaaaacatttacgggtcatttgtatgaaggtttgaaaaataggggaataagcacctttcaagatgataaaaggctagagcatggagattcaattccgaaagaactcttgagagctatcgaagagtctcaagttgcacttattgtcttctcaaagaattatgctacatctaggtggtgcttgaatgaactagtgaagatcatggaatgcaaagatgaagaaaatggacaaacagtcataccaatcttctatgatgtggatccatcacatgttcgaaaccaaagtgaaagctttggagcagcatttgccgaacatgaattaaagtataaggatgatgttgaggggatgcaaaaggtgcaaagatggagaaatgccctaactgttgccgcaaatctaaaaggatatgatatccgtgacgggttagttgaatacacataattacttttaatgaaaaaatggaataatttccattcataatttcattgattattatttagatggtagtgtatatacatttttattgttaagaaggcatagttctatcaatttaattagagaggatacataaaagtcctcctaaactatcaatcttcttctttatgtaggattgaatcagagaatattcaacagatcgtagactgcatctcttccaagtttcgcacaaatgcttattctttatcttttttgcaagatgttgtgggaataaacgatcacttagagaaactaaaatccaaacttcaaatggaaatcaacgatgttcggattttagggatctggggaataggcggagtcggtaaaacaacaatagcaaaagctatttttgatgctatatcttatcaatttaaagcttcctgttttcttgcagatgttaaagaaaatgcaaaaaagaataaactgcattctttacaaaatacccttctctctgaacttttaagagaaaaaaaaggttacgtcaataataagtatgatgggaagcgcatgattccgaacatactttgttctatgaaggtgctaattgtgcttgatgatatagatcacagtgagcatttggagtatttagcaggtgatgttggttggtttggtaatggcagcagagtcattataacaactagaaacaaacatttgatagagaaggatgatgcgatatacgaagtgtctacactacctgatcatgaagctatgcaattattcaataagcatgcttttaaaaaagaagatccagatgagagttttaagaagttctcattggaggtagtaaatcacgctaaaggccttcctttagccctcaaggtgtggggttctttattgcataaaaagtgtctaactttgtggagaaaaactgtagagcagataaagaaaaactctaattcagaaattgttaaaaaactcaaaataagttatgatgggttggagctcgaagagcaagagatatttctagatattgcatgtttattccgtggaaaaaaaagaaaagaagtcatgcaaattcttgagagttgtgactttggagctgaatacggattgaatgttctgattaataaatctcttgtgttcatctctgaaaataatatgattgaaatgcatgatttgattagagatatgggtagatatgtggtgaaaatgcaaaagcttccgaaaaaacgtagcagaatatgggatgttgaagatttcaaaaaagtgatgatagactatacagtaagtaagctaaacaatgcaataatatttaatttctaatttttatatttcaaagacgtataagccaatcaattccaattacttgttcctcttgcttcatattcttacaggtacgtcattttaggtttttactttactttattttattttatttttcttaataaaagaagcaaaagtaacatcaattgcctaacatagctaatcgtctatgaattagtgtttaatatgttttatccagtttctttcttaagattgcatgttatacacaccaataagtaaatagtttttaaagctaatatcacccacatatttaccctataaaaatcctcacataaaaaaatacaagtaagaagggtcaaatcataacataccaatcctcaaccaagaattcacttaatcattgatttgaaaaaataataattaaaagatgacaattatttgtattgttccaattacaatatttattctatcactgcattgaattttttatgatttgatgctaaagaacttctaaaaaaaaagtattattagaaagtataaatatttgcacaaaatatttcttgtaagcaacatgtgaagttatgattagttgactcatgatctttttatcaggggaccatgacagtggaagcaatccggctttgttgctttgaagaagaaatatgctttaataatgaggcaatgaaaaaaatgaaaagccttaggatattacacatagttgctactaataccaatttctttgcttcacgtccctcttcgaatcaccatgatgactctattgagtacctgtccaataacttgcgttggttagtctggaatgccttttcttggaaatcattgccagaaaattttaaaccagaaaagcttgttcatcttcaacttcgtgggagttcgctgcattatttatggaaggaaacagaggtaccattttatttaagctactttctaaggaaaagggtaaatatcctcaactttgcgatttgaaactgatatatcttttgtttaaaaaaatgatatatatactatgtcgtctaacaaatggtgcatatgtaccatttccgttaacgggtctgtatttattgaattaaaaaaataattataaaattgatttttaaaattcaaaaatatcatgtggctttaaaaaattaccccactcattttttttcccctctagacccaacccaaatgaataaaaacccaatccatcttctactcaaacataggatgagtttgagtcaggtctgaataaagagcgatgaggttctttagttggattaggtctagaggggtaaaaattgagtgagtaattttttaaaaaaccatgtgagtgtttagaaattaaaaaactaacttcaatttttttaaaatttaattgttaatgaaaataataaatatgcattatttgttaaacgttcatgttatatatgcatcattttatatttttaacaaaaggtatactcactccagatcacatagttaagatgtatatttgccccttttttctactttacatggttacttgtcctagaattatggtatttgtatttaaatgaaataagttattaattccataaactcaaattaatatatgttatacaatattgagttttcatattgttactatgaaaaagggctaaaattgtccttaaactaacaagtatagctcaattaatccttcatttgcttctgacaaccaaaacaccctgccatctattttctgatggatttaatgtaacatacacttttaaacaatcaagttaagttcgactttaaaaaaatagattaggtaagaatcactcattgatacgacgaaacccaacgtcatataaatgtaaaaattaaataagcaatttcttcaaaggcaatttaaccctattttgtcagattgaattgtcatgtcagctttttctattatttaaatggccctaacatgctcatgtggacaacattagcttggaggcattttttggtccaaaaaatagataacaagggtatttttggtataccaaaagcaaatgaaggataaaatggagctatttctgatagttcaagggcaatttagtcccttttctattaaaattataatatcgttatgtcttttttttcaaatcttgcctcattattttaactaagaagctcaatttaaaatttgatcattcaaatatttacctacaaaaatatgaatctcatcctaggttttatgtagtgggacaaaatgaatacttatcgaaggagttcattctatactactatcttcttgaatgatgattaaggtttcatatattggatttgggctaaaactataccatatctataggaataggttcattttggtcttcaaatatatataatatgaatatttttaatcccttcattttgctaaagtggagtacttttaatctccttaataaaatccgtttaaaagtaacagtgttaaactcaagtaacatccacatggctcttattctcttaaaacataaaatccattgggtaaattcattccccgtttaatagttgtctagatgccatatttttaatatttgatctacaccgacaactaaacactctcaccaaaagagaaaaatagataattaaaataaatagtcattacagctaaagaattaaaaaaaaaacataaattggggttaaaattatgccctctatttttcttttcactcaagcacctagaagagtgtattcactcttttccacatcaatgttcaagagataaatgatactccctccgtttcacaaagaatgacctccttttttttttagtcagtttaaaaaagaatgacctttttctatttttggtaacattttttttcacgtgacatgtttaaggccacaagattaaaggacaattttgtacatttgacataactttaatgtaggaccacagattcaaaagttttctttattttcttaaacgtcgtgtcaagtcaaactagaccattctttgtgaaattgagaaagtagtaaatagtcaagtatatataggggatatttaagataagtgtaacaatcaagtgtgtatcgaataaaacatgtcaagtctaaaggattatagataataaacacttttatttagtttctgaacaagtaatttcaatataaaaataaaataaaacattttctaatttcacttaaaatattttgttagactatcataatccaaatgtctttattcatttttggtctaaggggtaaattaaacacaaagataattctttctctattttgaatagcagcatttgccgtctctacgaaagctagatctctgcttttctaaaagcctagttcaaacaccagatttcacggggatgccaaatttggagtatttgaatctggagtactgtagtgaacttgaagaggttcacaattccctagcatattgcgaaaacctcattgagttaaatttgaattggtgtaacaagcttaggagatttccatgtgttaacatgaaatctcttgaatctatggatctacaatcgtgctataatttaatgaagacggagttagtgattctctcagcaaagggttggataaggaaattttcatcatctattcagtacctaactgatctcacaaacctagatttgagtggcatggaaaaccttgaagcacttccaagcagcattgttaagttgaaaggtttggtgaagctaaatgtgtcgtactgctttacaattaaaagcttgcccgaagagataggtgatttagaaaatttggaggaacttgatgctacatgtactctaatttcacgacctccttcttccattgtccgcttgaacaagcttaaatccttgaagtttgtaaaaattgaaacagaagatgaagtgtactttgtgtttcctccaattaatggcgggttactctcattggaaatcctgaagctcagttcctccaatttcatagatggaagaattccggaagatattggatacttatcctctttgataaagttgcatctcaagggagataattttgagcatttgcctcaaagcatagcccaacttggtgctcttcgagtcttatccttagaaggttgcaagaggcttacacagttgccagaatttccaccgcaattagatacaatatgtgcagattggcacaatgatttgatctgtaattcactatttcaaaatatctcatcattccagcatgacatctctgcttcagattccttgtcgttaagagtgtttacgagttgggggagtaatatccctagttggttccactatcagggaatggataaaagtgttttagtcaatttgcctgaaaactggtgtgtatcagataacttcttgggatttgctgtatgttactctggaagtttaattcaaaacatggctcaattgattattagttctgaagggatgccgtgtatcacccagaaacttggcttatacaattattcagaatgtattccagattgtgtgattcagtttttcttggtaccttttgctggcatatgggatacatctaacgcaaatggtaaaacaccaaatgactatgggcacattatgttatcttttcctgaagaattgaagaagtgtggacttcgtttgttctataaatatgaatctgagcttgttgagaccaatgatgaaccaccaacagaactttccatggggataaggaggatcagatacgacgatagtgaacatcatgaagaagccagttgttcctcttctaagaaacaaaggtcataagtagaggtatatatctcgatatgtatctaaacttctcgctttgctttctgttattaatagttcattttgatgctctttgtggttattataaaagaggaaatttttgttctaaatacaaatatctctcatgttatggcagggagtttgcaaaaacgatgaagcattaatctataagtgtgagacagtggaggaagctggagtatgaggtgaagttgaggttcgtcctccctcatgattgatctcttttggaccagaatacctagtttcactgtccaaaagaagtgtggattaactactgcaaatctacatggtagaagtagtttgtgcatacactgcggatggtcaatctagcacgtggattaagtagttcagatcgagcaactacattgttttttcatccagtgtctgggtacctgtcataggtttccacttaatgtaattcaaatgtcacattgttggaaccaattgacttaagccaattaaaaatatttttgtcaatcccttgttaccgaacaaatgttgtgctttaggaagttgtgaaaagggaaaatgaacaaatgatcccttgtgatttttttttatttgaataaacataatgaaagtttagcaaattgacaagtttacgaggttttggttattttaccttttatttataataatactacttttatttagtttcttgggtgtgcaatagtttgcgaaacagaagatggtactttgacaatatcgatgatttcgcgaaaacgggtggatgaaagaagcctcggaactgtttctagctgaccatccaaataccttaattgggtgttcatttgagtaaattttaggtttaaaaatcatattagcgctctatagctacagttttgttatttgtgtttcctagcaaactatatgccgtttgtataaaagcgtatatatgtatatcaattaaataattgtatatatgtaactactatgtatatgtatcaattattgtgtttgtatatctgcataaaatttaaatttgtatgcaattgaatcgaaataaaacatttgtatatcaaatatctctcgctttatacaacacaaattatacattgtaattgtattgtgtttgtataaagcaagaaagagagaaaggcaaaaaagaattggtagggaaatatttgtatttatataattataagtgtataggacgaaaatatatgtatttgcatttgtatatatacaattttctctcgctttatacaaaaacaaacacaatttatacatttgtattgtataaaatgagagaggcgagggagagtggcgagcgagatttctggggagagaggctaatggcaaagtgtttgctacgaattagaattaaataaaactgtagttataacatttatttcgaattaataatttgttatttcatataatttccctataaagtatgataaatagctggccttccttagatttatgggccttttgaagaagtatcaacgttgctggactcttgggcttttaaggcccaagaaggtgggcttactgctagaaaatgaagtagttaaagctaaaagattggtgtctgtaatatatgtagttaatatatgggaaaattatgcggttaagcaaatttatactacttaattactcatcatagctatagtatgctataattaccactcgcgactaacattatacattaattacgtgggctgacttcgagtttgtataattagccacgtttgtatatgtataattcgctagaatatacaaatacatatgtataatatacaattatctatctgatatacatatacattcacctctctcccactctctgccctcaatcttgcttgccatatatacaaatgcgtatgtataatatacaattatctaactgatatacatatataattcacctctctcccactctctgccctctctcgcttgcctctctcctccctctcccaatctcgcttgccatatatacaaatacatatgtataatatacaattatctaaccgatatacttatacaattcacctctctccaactcttagtcctctctctcccagtctcgctcgcctctctcctccccatgacatgtagcaacgaattgtaattatcaaactacagtcatggagagtaattaggctatttttgattggctatatgtgaaagtttctcttaatatatcacgatctcttgagttgtgggctttaagggagtgaattctttac

(19) United States
(12) Patent Application Publication
(10) Pub. No.: US 2012/0291157 A1
(43) Pub. Date: Nov. 15, 2012

(54) GPAV GENE RESISTANT TO NEMATODES IN THE SOLANACEA

(76) Inventors: Bernard Caromel, Avignon (FR); Jawad Aarrouf, Le Pontet (FR); Laura Chauvin, Landerneau (FR); Marie-Claire Kerlan, Riec sur Belon (FR); Véronique Lefebvre, Montafavet (FR); Adrien Speck, Marseille (FR)

(21) Appl. No.: 13/519,886

(22) PCT Filed: Dec. 31, 2010
(86) PCT No.: PCT/EP2010/070952

§ 371 Date: Jun. 28, 2012

(30) Foreign Application Priority Data
Dec. 31, 2009 (FR) 09/06432

(51) Int. Cl. A01H 5/00; A01H 5/10; C12N 15/82; C07H 21/04; C12Q 1/68; C12N 5/10

(52) U.S. Cl. 800/279; 435/6.12; 435/419; 435/320.1; 536/23.6; 800/298; 800/301; 800/317; 800/317.2

(57) ABSTRACT

The invention relates to a GpaV gene providing resistance to nematodes in plants belonging to the nightshade family, and in particular potatoes (Solarium tuberosum et Solarium phureja).

The invention relates to the GpaV gene, which confers nematode resistance in plants of the Solanaceae family.

It is understood today that the activation of plant defense mechanisms results from a cascade of events during which higher plants and pathogenic agents exchange molecular signals. Signals that trigger defense mechanisms are called elicitors. Recognition by the host cell of an elicitor produced by the pathogenic agent or by the plant constitutes a prior and necessary step for specific gene activation (gene-for-gene recognition); others are general (nonspecific recognition). According to the behavior of the host, total resistance can be distinguished from partial resistance and, according to the spectrum of action of the resistance gene, specific (or vertical) resistance can be distinguished from general (or horizontal) resistance. The presence of resistance genes can limit, delay or prevent the course of the pathogenic agent's infection cycle in the plant.

Plants react very early to attempts at invasion by pathogenic agents by aiming primarily at preventing or stopping colonization of the pathogen. The cell wall is a natural physical barrier that is highly effective against pests and diseases that synthesize enzymes and compounds capable of breaking the cell wall down. During infection by a pathogenic agent, this wall is reinforced by deposits of phenolic compounds (lignins), esters such as suberin, polysaccharides such as callose and by the accumulation of hydroxyproline-rich glycoproteins.

The biochemical responses are: (1) synthesis of phytoalexins (antibiotic compounds), (2) synthesis and accumulation of low-molecular and weight phenolic compounds and of proteins in the cell wall, (3) synthesis of pathogenesis-related (PR) proteins. These compounds are synthesized in response to recognition of the pathogenic agent to inhibit its growth and its development (lytic enzymes, phytoalexins) but also to limit its propagation in the plant.

Implementation of defense mechanisms results from transcriptional activation of a large number of genes. These genes code for enzymes of the phenylpropanoid biosynthetic pathway or for defense proteins, some of which have known hydrolytic activities (chitinase, glucanases, RNases, protease inhibitors). The molecular dissection of defense gene promoters revealed cis-regulatory regions as well as trans-regulatory elements.

The resistance phenomenon can be due to the effect of resistance alleles of a single gene (monogenic resistance), or to the combined effect of alleles of several genes (polygenic resistance). Monogenic resistance results from a specific interaction between a resistance gene of the host plant and an avirulence gene of the pathogen. This interaction can be direct or indirect. However, monogenic resistances often have the disadvantage of quickly being circumvented by the parasite and are in this case not very long-lasting. Polygenic resistances are considered to be more long-lasting, but difficult to analyze and exploit. Several loci govern this type of resistance, namely quantitative trait loci (QTLs). In contrast with monogenic resistances, resistances conferred by QTLs are often non-pathotype specific, and lead to a slowing of the development of the disease.

The molecular marking of genomic regions involved in resistance is possible today using molecular markers that make it possible to establish genetic linkages (genetic map). Among these linkages are located regions associated with phenotypic variation of the characteristic. These regions are commonly called QTL in the case of quantitative or polygenic resistance. This use of molecular markers should make it possible in the long term to separately and specifically select each QTL on the basis of its effect, origin and mode of action.

Potato (Solanum tuberosum ssp. tuberosum) belongs to the family of Solanaceae. This family also includes other widely-cultivated plant species such as tomato (Solanum lycopersicum), pepper (Capsicum sp.) and eggplant (Solanum melongena, also known as aubergine). Potato is one of the world's largest crops. This tuber is indeed produced in more than 130 countries. With production of more than 320 million tons in 2007, it is the world's third largest food crop, after wheat and rice (data: FAO, 2007). In France, the quantity of potato produced is estimated to be 4,440,000 tons in 2006, including one million tons intended for processing and two million tons sold fresh in France (data: FAO, 2007).

Two species of cyst nematodes attack potatoes: Globodera pallida and G. rostochiensis. These two species are listed as quarantine pests. Lost potato production due to nematodes is estimated to be 12.2% of worldwide production. If nematode population levels are very high, 80% of the harvest can be lost.

Cyst nematodes are worms of very small size (less than 1 mm). Their cysts, which result from transformation of females after fertilization, are visible to the naked eye on the roots. Females are white when they appear on the root surface; those of G. pallida remain white whereas those of G. rostochiensis move through a golden yellow phase. When the females are fully developed they die; their skin hardens, becomes brown and is transformed into a protective envelope, the cyst. This cyst can contain more than 1,000 larvae; it is thus the essential element that ensures preservation and dispersion of the species. This is thus its resistant form. Thanks to its chitin-rich cell wall, it is able to tolerate hostile environmental conditions and can, in this form, preserve itself several years in the ground. The cyst contains second-stage juvenile larvae J2, which is the infective form. J2 in the cyst are in diapause. The lifting of this latency state is stimulated by root exudates secreted by a potential host plant. J2 nematodes first attach to roots and then penetrate and develop within the root to induce formation of their feeding site, the syncytium. This is a very large multinucleate cell, with dense cytoplasm, resulting from the fusion of several tens of adjacent cells. The syncytium has an important function during the growth of juveniles, which under favorable conditions develop preferentially into females (more than 90%). In the case of unfavorable conditions (competition between nematodes too high, poor physiological condition of the attacked plant, presence of certain resistance genes), they develop preferentially into males or remain stuck in a larval stage.

A plant's resistance to nematodes has been defined as the aptitude of the host plant to reduce or prevent nematode reproduction. In the present case, the genes involved in cyst nematode resistance described hitherto in Solanaceae oppose neither the penetration nor the migration of juveniles into the root (Caromel et al., 2004). The expression of resistance appears after initiation of the syncytium, by inducing necrosis in surrounding cells, thus preventing its functioning as transfer cell. The nematodes are then deprived of food, which frequently results in inversion of the population's sex ratio (percentages of males and females), typically observed in sensitive plants. The greater the extent of necrosis, the less the syncytium develops. In the case of extremely serious necrosis, most of the nematodes remain stuck in a juvenile stage (Mugniéry et al., 2001; Caromel et al., 2005). With the exception of the Gpa2 gene, which confers resistance to only some G. pallida populations (Rouppe van der Voort et al., 1997), no high-level monogenic resistance has been detected in potato.

On the other hand, QTL mapping of G. pallida resistance, from sources of natural resistance belonging to three wild species of potato, S. sparsipilum (Caromel et al., 2005), S. spegazzinii (Caromel et al., 2003; Kreike et al., 1994) and S. vernei (Bryan et al., 2002; Rouppe van der Voort et al., 1998, 2000), showed that the expression of a strong-effect QTL, located in a collinear position on chromosome V (locus GpaV) of these three species, as well as the expression of a weak-effect QTL, was essential for obtaining a high level of resistance. The latest mapping results locate the GpaV resistance gene or genes in a region of approximately 5 cM on S. sparsipilum chromosome 5.

In S. sparsipilum, the combined effect of the GpaVₛₚₗ QTL, mapped on chromosome V, and of the GpaXIₛₚₗ QTL mapped on chromosome XI, considerably reduces cyst nematode development. The expression of these QTLs enables the plant to develop necrosis at the parasite-infected root, and thus prevents the syncytium from developing. The result is that the nematodes are no longer properly nourished and less than 1% will develop into females (Caromel et al., 2005).

The present invention thus relates to the complete genome sequence of a GpaV gene resistance allele conferring a strong resistance to nematodes in complementation tests.

### SUMMARY OF THE INVENTION

The invention relates to isolated polynucleotides selected from the following polynucleotides:
the polynucleotide of SEQ ID No. 1, the polynucleotide of SEQ ID No. 3, the polynucleotide of SEQ ID No. 5, the polynucleotide of SEQ ID No. 7, the polynucleotide of SEQ ID No. 9 and the polynucleotide of SEQ ID No. 11;
a polynucleotide coding for the polypeptide of SEQ ID No. 2, the polypeptide of SEQ ID No. 4, the polypeptide of SEQ ID No. 6, the polypeptide of SEQ ID No. 8, the polypeptide of SEQ ID No. 10 or the polypeptide of SEQ ID No. 12.

The invention also relates to an isolated polynucleotide conferring to plants of the Solanaceae family resistance to Globodera, wherein said isolated polynucleotide is selected from:
a polynucleotide with at least 80% homology with the polynucleotide of SEQ ID No. 1, the polynucleotide of SEQ ID No. 3, the polynucleotide of SEQ ID No. 5, the polynucleotide of SEQ ID No. 7, the polynucleotide of SEQ ID No. 9 or the polynucleotide of SEQ ID No. 11;
a fragment of a polynucleotide of SEQ ID No. 1, of a polynucleotide of SEQ ID No. 3, of a polynucleotide of SEQ ID No. 5, of a polynucleotide of SEQ ID No. 7, of a polynucleotide of SEQ ID No. 9 or of a polynucleotide of SEQ ID No. 11.

Preferably, the plants are selected from plants of the species Solanum tuberosum L., Solanum phureja, Solanum lycopersicum L., Solanum melongena L. and Nicotiana tabacum and plants of the genus Capsicum.

Preferably, the nematodes are selected from Globodera pallida, Globodera rostochiensis, Globodera tabacum ssp. tabacum, ssp. virginiae and ssp. solanacearum, and Globodera mexicana.

The invention also relates to expression cassettes comprising in the direction of transcription:
a functional promoter in a host organism,
an isolated polynucleotide of the invention;
a functional terminator sequence in the same host organism.

Advantageously, the functional promoter in a host organism is selected from CaMV 35S, T-DNA promoters, the promoters of genes coding for ubiquitins, promoters expressed specifically in roots and the promoter from position 1 to position 1657 of SEQ ID No. 1.

The present invention also relates to a vector comprising a polynucleotide of the invention or an expression cassette of the invention.

The present invention also relates to a host cell transformed with a polynucleotide, expression cassette or vector of the invention.

Preferentially, the transformed host cell is selected from plant cells and plant cell protoplasts.

The invention also relates to a host organism comprising at least one transformed cell of the invention.

The invention also relates to a host organism transformed with a polynucleotide, expression cassette or vector of the invention.

The host organism is a nonhuman host organism.

Advantageously, the transformed host organism is selected from plants, seeds and plant tissue.

The invention also relates to a transformed plant expressing a polynucleotide, expression cassette or vector of the invention.

The invention also relates to a plant expressing a polypeptide of one of SEQ ID Nos. 2, 4, 6, 8, 10 or 12.

Preferably, the transformed plants belong to the family of Solanaceae. More preferentially, the transformed plants are selected from plants of the species Solanum tuberosum L., Solanum phureja, Solanum lycopersicum L., Solanum melongena L. and Nicotiana tabacum and plants of the genus Capsicum.

The invention also relates to plants selected from plants of the species Solanum tuberosum L., Solanum phureja, Solanum lycopersicum L., Solanum melongena L. and Nicotiana tabacum and plants of the genus Capsicum comprising or expressing a polynucleotide or a polypeptide of the invention.

Preferably, the plant is selected from crop potatoes and it comprises or expresses a polynucleotide of the invention or a polypeptide of the invention.

The invention also relates to a method for conferring Globodera nematode resistance to a plant of the Solanaceae family, comprising the following steps:
transforming the plant with a polynucleotide, expression cassette or vector of the invention;
selecting a plant resistant to Globodera nematodes.

The invention also relates to a method for rendering resistant to Globodera nematodes a Solanum tuberosum L. subsp. tuberosum, Solanum tuberosum L. subsp. andigena, or Solanum phureja plant, comprising the following steps:
introgression of a segment of Solanum sparsipilum genomic DNA comprising a polynucleotide of the invention in a Solanum tuberosum L. subsp. tuberosum, Solanum tuberosum L. subsp. andigena, or Solanum phureja plant;
selecting a Solanum tuberosum L. subsp. Tuberosum, Solanum tuberosum L. subsp. andigena, or Solanum phureja plant resistant to Globodera nematodes, with molecular markers derived from SEQ ID No. 1.

The invention finally relates to the use of polynucleotide primers or probes derived from SEQ ID No. 1 for the detection of plants of the Solanaceae family nematode-resistant.

Sequence Listing
SEQ ID No. 1: Genome sequence of the GpaV gene
SEQ ID No. 2: GpaV protein
SEQ ID No. 3: cDNA 1
SEQ ID No. 4: Protein coded by cDNA 1
SEQ ID No. 5: cDNA B6
SEQ ID No. 6: Protein coded by cDNA B6
SEQ ID No. 7: cDNA 8
SEQ ID No. 8: Protein coded by cDNA 8
SEQ ID No. 9: cDNA 9
SEQ ID No. 10: Protein coded by cDNA 9
SEQ ID No. 11: cDNA H1
SEQ ID No. 12: Protein coded by cDNA H1
SEQ ID No. 13: Primer MS063_{—}2F
SEQ ID No. 14: Primer MS063_{—}2R
SEQ ID No. 15: Primer MS092_F
SEQ ID No. 16: Primer MS092_R
SEQ ID No. 17: Primer ASC102_F
SEQ ID No. 18: Primer ASC102_R
SEQ ID No. 19: Primer ASC231_F
SEQ ID No. 20: Primer ASC231_R
SEQ ID No. 21: Primer ASC240_F
SEQ ID No. 22: Primer ASC240_R
SEQ ID No. 23: Primer Z751_{—}2F
SEQ ID No. 24: Primer Z751_{—}2R
SEQ ID No. 25: Primer Z1505_{—}6F
SEQ ID No. 26: Primer Z1505_R
SEQ ID No. 27: Primer Z1505_{—}8F
SEQ ID No. 28: Primer Z1505_{—}4R
SEQ ID No. 29: Primer Q63F
SEQ ID No. 30: Primer Q63R
SEQ ID No. 31: Primer Z1505_{—}5R
SEQ ID No. 32: Primer 23461 F
SEQ ID No. 33: Primer 23461 R
SEQ ID No. 34: Sensitive allele of Solanum sparsipilum clone spl329.18
SEQ ID No. 35: Sensitive allele of Solanum sparsipilum clone spl504.5
SEQ ID No. 36: Sensitive allele of Solanum tuberosum clones Caspar H3 and Rosa H1

### DESCRIPTION OF THE INVENTION

The invention relates to the GpaV nematode-resistance gene, isolated from Solanum sparsipilum, a wild Solanaceae species related to the potato. The invention relates to the polynucleotide sequence of this gene comprising the coding part of the gene as well as regulatory sequences located upstream and downstream from these coding sequences. The invention also relates to expression cassettes comprising the coding part of this gene, vectors as well as polypeptides coded by the GpaV gene.

The GpaV gene confers to plants of the Solanaceae family a high level of nematode resistance. With the GpaV resistance gene, it is thus now possible to confer nematode resistance to other plants of the Solanaceae family and in particular to plants of widely cultivated plant species.

The expressions “resistance to Globodera nematodes” and “Globodera nematode resistance” refer to the resistance of plants, in particular plants of the Solanaceae family, to nematodes of the genus Globodera. The most commonly practiced tests are tests on plants in pots in which the plants are inoculated either with cysts or with already-hatched J2. After cultivation, the number of cysts neoformed on the roots are counted and compared with the number of cysts neoformed on a sensitive control (for example, the Desiree variety sensitive to G. pallida and G. rostochiensis). The procedure for carrying out the resistance test is described in European Union Directive 2007/33/EC.

The G. pallida resistance test makes it possible to measure the number of cysts neoformed on a potato plant after one complete nematode life cycle. It is carried out on 4 plants (4 repetitions) of each genotype with cysts of the Chavornay population, which corresponds to a Pa3 pathotype in the scheme of Kort et al. (1977). The tubers are planted individually in a pot containing 400 grams of a mixture of soil-based compost and sandy loam, to which 10 G. pallida cysts are added. This number of cysts is sufficient to obtain, after hatching, 5 to 10 nematode larvae per gram of soil. The plants are cultivated in a greenhouse. A complete cultivation cycle is carried out in order to allow the nematodes time to develop and to encyst. After four months of cultivation, the contents of each pot are washed and filtered in preparation for the counting of neoformed cysts. The average number of nematodes found on each potato genotype is compared to the average number of nematodes found on the control variety Desiree, sensitive to G. pallida, following the protocol of Council Directive 2007/33/EC of 11 Jun. 2007, published in the Official Journal of the European Union on 16 Jun. 2007.

The expressions “resistance to Globodera nematodes,” “Globodera nematode resistance” and “Globodera resistance” refer to the resistance of plants of the Solanaceae family, for which less than 200 or less than 100 neoformed cysts, preferably between 0 and 100 neoformed cysts, more preferentially between 0 and 80 neoformed cysts are obtained after carrying out the test according to the protocol above. Typically, under the same conditions, more than 400 cysts on the sensitive variety Desiree are observed.

The present invention relates to resistance to nematodes of the genus Globodera, which are well-known devastator of plants of the Solanaceae family. Particular mention may be made of Globodera pallida and Globodera rostochiensis which infest potato, tomato and eggplant, as well as Globodera mexicana which infects tomato and Solanum related to potato and Globodera tabacum (including the subspecies Globodera tabacum tabacum, Globodera tabacum virginiae and Globodera tabacum solanacearum) which primarily infect tobacco but also peppers and certain species related to potato.

The GpaV gene of Solanum sparsipilum confers Globodera nematode resistance to plants of the Solanaceae family and in particular plants of the species Solanum tuberosum L., Solanum phureja, Solanum lycopersicum L., Solanum melongena L. and Nicotiana tabacum as well as plants belonging to the genus Capsicum.

Advantageously, the GpaV gene confers nematode resistance to potato (Solanum tuberosum L. and Solanum phureja) notably including many commercial cultivars. The cultivated potatoes include both subspecies, Solanum tuberosum L. subsp. tuberosum and Solanum tuberosum L. subsp. andigenum, as well as Solanum phureja. In a preferred embodiment, the GpaV gene confers nematode resistance to cultivated potatoes of the subspecies Solanum tuberosum L. subsp. tuberosum.

Polynucleotides

The invention thus relates to the polynucleotide sequence of the GpaV gene represented by SEQ ID No. 1. SEQ ID No. 1 also comprises sequences upstream and downstream from the GpaV gene coding sequence. In particular, position 1 to position 1657 of SEQ ID No. 1 notably contains the GpaV nematode-resistance gene promoter.

The invention thus also relates to the GpaV nematode-resistance gene promoter and in particular to the polynucleotide having the sequence from position 1 to position 1657 of SEQ ID No. 1.

The GpaV gene coding sequence corresponds to the following positions on SEQ ID No. 1: 1822-2330, 2526-3615, 4227-4532 and 6844-8322. This coding sequence is represented by SEQ ID No. 3.

Preferably, nematode resistance is obtained by transformation of plants of the Solanaceae family with the polynucleotide of SEQ ID No. 1 or by introgression of a genomic fragment comprising the polynucleotide of SEQ ID No. 1 in plants of the Solanaceae family.

Finally, the genomic fragment of Solanum sparsipilum comprising the polynucleotide of SEQ ID No. 1 introgressed in a plant of interest preferably has a size smaller than 20 kbp, 50 kbp, 200 kbp, 250 kbp, 500 kbp or 1 Mbp.

In another embodiment, nematode resistance is obtained by transformation of plants of the Solanaceae family and expression of the polynucleotide of SEQ ID No. 3 or by expression of the polypeptide of SEQ ID No. 2 in Solanaceae.

The GpaV gene transcript is susceptible to alternative splicing and various messenger RNAs corresponding to the GpaV gene have been identified. The SEQ ID Nos. 5, 7, 9 and 11 represent the preferred cDNA (cDNA B6, cDNA 8, cDNA 9 and cDNA H1) corresponding to various messenger RNA.

In another embodiment, nematode resistance is obtained by transformation of plants of the Solanaceae family and expression of the polynucleotide of SEQ ID Nos. 5, 7, 9 or 11 or by expression of the polypeptide of SEQ ID No. 6, 8, 10 or 12 in Solanaceae.

The GpaV gene comprises exons at the following positions on SEQ ID No. 1: 1658-2330 (exon 1), 2526-3615 (exon 2), 4227-4532 (exon 3), 6844-8331 (exon 4) and 8465-8811 (exon 5).

Various possibilities of coding sequences resulting from various types of alternative splicing have been identified. These sequences were constructed from sequences obtained by RACE and RT-PCR. The 5′ and 3′ untranslated regions UTR were not represented: the sequences begin with ATG (exon 1) and terminate at the first stop codon encountered (depending on the shift in the reading frame due to alternative splicing, the stop codon can be in an exon or an intron). These additional cDNA correspond to the following positions on the exons/introns of SEQ ID No. 1: cDNA_{—}2 (E1: 1-509, E2: 510-1599, E4: 1600-3081), cDNA_{—}3 (E1: 1-509, E3: 510-519), cDNA_{—}4 (E1: 1-509, E4: 510-549), cDNA_{—}5 (E1: 1-509, δE2: 510-633, E3: 634-939, E4: 940-2421), cDNA_{—}6 (E1: 1-509, δE2: 510-633, E4: 634-2115), cDNA_{—}7 (E1: 1-509, I1: 510-528), cDNA_{—}8 (E1: 1-509, E2: 510-1599, I2: 1600-1638), cDNA_{—}9 (E1: 1-509, E2: 510-1599, E3: 1600-1905, I3: 1906-1992), cDNA_{—}10 (E1: 1-509, δE2: 510-633, I2: 634-672) and cDNA_{—}11 (E1: 1-509, δE2: 510-633, E3: 634-939, I3: 940-1026).

The invention also relates to the polynucleotides corresponding to these various cDNA and to the polypeptides coded by these cDNA.

In another embodiment, nematode resistance is obtained by transformation of plants of the Solanaceae family and expression of a polynucleotide corresponding to one of the various cDNA above or by expression of a polypeptide coded by one of the cDNA above.

The invention relates to an isolated polynucleotide selected from the following polynucleotides:
the polynucleotide of SEQ ID No. 1, the polynucleotide of SEQ ID No. 3, the polynucleotide of SEQ ID No. 5, the polynucleotide of SEQ ID No. 7, the polynucleotide of SEQ ID No. 9 and the polynucleotide of SEQ ID No. 11;
a polynucleotide coding for the polypeptide of SEQ ID No. 2, the polypeptide of SEQ ID No. 4, the polypeptide of SEQ ID No. 6, the polypeptide of SEQ ID No. 8, the polypeptide of SEQ ID No. 10 or the polypeptide of SEQ ID No. 12.

The invention also relates to an isolated polynucleotide conferring Globodera nematode resistance to plants of the Solanaceae family, wherein said isolated polynucleotide is selected from:
a polynucleotide with at least 80% homology with the polynucleotide of SEQ ID No. 1, the polynucleotide of SEQ ID No. 3, the polynucleotide of SEQ ID No. 5, the polynucleotide of SEQ ID NO. 7, the polynucleotide of SEQ ID No. 9 or the polynucleotide of SEQ ID No. 11;
a fragment of a polynucleotide of SEQ ID No. 1, of a polynucleotide of SEQ ID No. 3, of a polynucleotide of SEQ ID No. 5, of a polynucleotide of SEQ ID No. 7, of a polynucleotide of SEQ ID No. 9 or of a polynucleotide of SEQ ID No. 11.

Preferably, the polynucleotides of the present invention are isolated from a plant resistant to nematodes of the species Solanum sparsipilum. These polynucleotides have homology with one of the polynucleotides of SEQ ID Nos. 1, 3, 5, 7, 9 or 11, and typically correspond to other resistance alleles of the Solanum sparsipilum GpaV gene.

In other embodiments, the polynucleotides of the present invention conferring Globodera nematode resistance to plants of the Solanaceae family are isolated from plants of the species Solanum vernei or Solanum spegazzinii. These isolated polynucleotides from Solanum vernei or Solanum spegazzinii typically code for orthologs of the GpaV gene of SEQ ID No. 1. These orthologous genes can be isolated from Solanum vernei or Solanum spegazzinii using polynucleotides derived from SEQ ID No. 1 as probes or primers.

The invention thus also relates to genes or polynucleotides orthologous to the GpaV gene of Solanum sparsipilum in Solanum vernei or Solanum spegazzinii. These genes or polynucleotides coding for a resistance allele preferentially with at least 75%, 80%, 85%, 90%, 95%, 98% and preferably at least 99% identity over their entire length with one of the polynucleotides of SEQ ID Nos. 1, 3, 5, 7, 9 or 11.

Preferably, the polynucleotides of the present invention confer Globodera nematode resistance to plants of the species Solanum tuberosum L. (ssp. tuberosum and andigena), Solanum phureja, Solanum lycopersicum L., Solanum melongena L. and Nicotiana tabacum and plants of the genus Capsicum.

Preferentially, polypeptides of the present invention confer resistance to the nematodes Globodera pallida and Globodera rostochiensis, Globodera tabacum ssp. tabacum, Globodera tabacum ssp. virginiae and Globodera tabacum ssp. solanacearum, and Globodera mexicana.

According to the present invention, “polynucleotide” refers to a single-stranded DNA or RNA chain or the complement thereof, or a complementary or genomic double-stranded DNA chain. Preferably, the polynucleotides of the invention are DNA, notably double-stranded DNA. The term “polynucleotide” also refers to modified polynucleotides.

The polynucleotides of the present invention are isolated or purified from their natural environment. Preferably, the polynucleotides of the present invention can be prepared by standard molecular biology techniques as described by Sambrook et al. (Molecular Cloning: A Laboratory Manual, 1989) or by chemical synthesis.

In a first embodiment, the invention relates to the polynucleotides of SEQ ID Nos. 1, 3, 5, 7, 9 and 11.

The invention also relates to polynucleotides with at least 75%, 80%, 85%, 90%, 95%, 98% and preferably at least 99% identity with one of the polynucleotides of SEQ ID Nos. 1, 3, 5, 7, 9 or 11.

Preferably, the invention relates to polynucleotides with at least 75%, 80%, 85%, 90%, 95%, 98% and preferably at least 99% identity over their entire length with one of the polynucleotides of SEQ ID Nos. 1, 3, 5, 7, 9 or 11.

The invention also relates to polynucleotides with at least 75%, 80%, 85%, 90%, 95%, 98% and preferably at least 99% homology with one of the polynucleotides of SEQ ID Nos. 1, 3, 5, 7, 9 or 11.

The invention also relates to polynucleotides with at least 75%, 80%, 85%, 90%, 95%, 98% and preferably at least 99% homology over their entire length with one of the polynucleotides of SEQ ID Nos. 1, 3, 5, 7, 9 or 11.

The invention also relates to fragments of at least 500 bp, 1 kbp, 1.5 kbp, 2 kbp or 2.5 kbp of the polynucleotides of SEQ ID Nos. 1, 3, 5, 7, 9 or 11.

The expressions “fragment” of a polynucleotide and “polynucleotide fragment” refer to a polynucleotide comprising part but not all of the polynucleotide from which it is derived.

Preferably, these polynucleotides confer nematode resistance to plants of the Solanaceae family when these polynucleotides are introduced and expressed in these plants.

The expression “identical nucleotides” refers to nucleotides that are invariant or unchanged between two sequences. These polynucleotides can have a deletion, an addition or a substitution of at least one nucleotide in relation to the reference polynucleotide.

The term “homology” refers to the measurement of resemblance between nucleic sequences. These polynucleotides can have a deletion, an addition or a substitution of at least one nucleotide in relation to the reference polynucleotide. The percent homology between two sequences, quantified by a score, is based on the percent identities and/or conservative substitutions of the sequences.

Methods for measuring and identifying the degree of identity and the degree of homology between nucleic acid sequences are well-known to the person skilled in the art. The lalign program (http://www.ch.embnet.org/software/LALIGN_form.html), for example, can be employed using the “global” alignment method and the default parameters, except for the “DNA” scoring matrix which will be selected for nucleotide sequences. This program calculates the degree of identity for the totality of the sequence. The BLAST program suite (http://blast.ncbi.nlm.nih.gov/Blast.cgi) makes it possible to rapidly identify genes with high homology with all or part of the sequence tested (QUERY). The program gives the percentage of identity for the homologous portions of sequences (local alignment).

Preferentially, the polynucleotides with a degree of homology with a reference polynucleotide conserve the function of the reference sequence. In the present case, the polynucleotides confer nematode resistance to plants of the Solanaceae family. The applicable tests of resistance are notably described above and in the examples.

The invention also relates to polynucleotides capable of hybridizing selectively with one of the polynucleotides of SEQ ID Nos. 1, 3, 5, 7, 9 or 11.

Preferably, the selective hybridization is carried out under conditions of moderate stringency and preferentially under conditions of high stringency.

In the context of the invention, the expression “sequence capable of hybridizing selectively” refers to sequences that hybridize with the reference sequence at a level significantly greater than background noise. The level of the signal generated by the interaction between the sequence capable of hybridizing selectively and the reference sequences is generally 10 times, preferably 100 times more intense than that of the interaction of other DNA sequences generating background noise. The stringent hybridization conditions enabling selective hybridization are well-known to the person skilled in the art. In general, the hybridization and washing temperature is at least 5° C. lower than the Tm of the reference sequence at a given pH and for a given ionic strength. Typically, the hybridization temperature is at least 30° C. for a polynucleotide of 15 to 50 nucleotides and at least 60° C. for a polynucleotide of more than 50 nucleotides. As an example, hybridization is carried out in the following buffer: 6×SSC, 50 mM Tris-HCl (pH 7.5), 1 mM EDTA, 0.02% PVP, 0.02% Ficoll, 0.02% BSA, 500 μg/ml denatured salmon sperm DNA. Washings, for example, are carried out successively with low stringency in 2×SSC, 0.1% SDS buffer, with moderate stringency in 0.5×SSC, 0.1% SDS buffer and with high stringency in 0.1×SSC, 0.1% SDS buffer. Hybridization can of course be carried out according to other common methods well-known to the person skilled in the art (see in particular Sambrook et al., Molecular Cloning: A Laboratory Manual, 1989).

Preferably, the polynucleotides being hybridized selectively to a reference polynucleotide conserve the function of the reference sequence.

The invention relates in a general way to the polynucleotides coding for the polypeptides of the invention. Due to genetic code degeneration, different polynucleotides can code for the same polypeptide.

The GpaV gene can be expressed in plants of the Solanaceae family from its homologous regulatory sequences notably for overexpression in Solanum tuberosum L. Thus, the GpaV gene can be expressed in a plant of the Solanaceae family under the control of the promoter of SEQ ID No. 1 of the present invention or under the control of a heterologous promoter.

The transformation and expression of the polynucleotides of the invention in plants of the Solanaceae family confer to the latter a resistance to nematodes and in particular to Globodera as described above.

The polynucleotide of SEQ ID No. 1 codes for a polypeptide comprising TIR, NBS and LRR domains. Preferably, the polynucleotides of the present invention code for a polypeptide comprising at least one TIR domain.

Polypeptides

The invention also relates to polypeptides whose expression in plants of the Solanaceae family confers Globodera nematode resistance.

The invention thus relates to polypeptides of SEQ ID Nos. 2, 4, 6, 8, 10 and 12. The invention also relates to polypeptides with at least 80%, 85%, 90%, 95%, 98% and preferentially at least 99% amino acids identical to one of the polypeptides of SEQ ID Nos. 2, 4, 6, 8, 10 and 12.

The term “identical amino acids” refers to amino acids that are invariant or unchanged between two sequences. These polypeptides can have a deletion, addition or substitution of at least one amino acid in relation to the reference polypeptide.

The invention also relates to polypeptides with at least 80%, 85%, 90%, 95%, 98% and preferentially at least 99% similarity with one of the polypeptides of SEQ ID Nos. 2, 4, 6, 8, 10 and 12.

The term “similarity” refers to the measurement of resemblance between protein sequences. These polypeptides can have a deletion, addition or substitution of at least one amino acid in relation to the reference polypeptide. The degree of similarity between two sequences, quantified by a score, is based on the percent identities and/or conservative substitutions of the sequences.

Methods for measuring and identifying the degree of identity and the degree of similarity between polypeptides are known to the person skilled in the art. To calculate the degree of identity for the entire sequence (global alignment), the lalign program (http://www.ch.embnet.org/software/LALIGN_form.html) can be employed, for example, using the global alignment method and the default settings. The BlastP program (http://blast.ncbi.nlm.nih.gov/Blast.cgi) makes it possible to rapidly identify genes with strong homology with all or part of the sequence tested (QUERY). The program gives percent identity and similarity for homologous sequence ranges (local alignment).

The polypeptides of the invention are isolated or purified from their natural environment.

The polypeptides of the present invention, when they are expressed in a plant of the family of Solanaceae, confer resistance to nematodes and in particular to Globodera as described above.

The polypeptide of SEQ ID No. 2 comprises the TIR, NBS and LRR domains. Preferably, the polypeptides of the present invention comprise at least the TIR domain.

Expression Cassettes

According to one embodiment of the invention, a polynucleotide coding for a polypeptide of the invention is inserted into an expression cassette using cloning techniques well-known to the person skilled in the art. This expression cassette comprises the elements necessary for the transcription and translation of sequences coding for the polypeptides of the invention.

Advantageously, this expression cassette comprises both elements for enabling a host cell or host organism to produce a polypeptide and elements required to regulate this expression.

Typically, these expression cassettes comprise in the direction of transcription:
a functional promoter in a host organism;
a polynucleotide of the invention;
a functional termination sequence in the same host organism.

Any type of promoter sequence can be used in the expression cassettes of the invention. The choice of promoter will depend notably on the host organism chosen to express the GpaV gene. Preferably, the expression cassettes of the present invention are for expression of the polynucleotides or polypeptides of the present invention in plants, seeds, plant tissues and plant cells, and more particularly for expression in plants of the Solanaceae family. Certain promoters enable constitutive expression whereas other promoters are in contrast inducible. Among functional promoters in plants, mention may be made of CaMV 35S promoters, T-DNA promoters, promoters of genes coding for ubiquitins (Garbarinov at al. 1994, 1995), promoters expressed specifically in roots such as Tob, RB7 and SIREO (Opperman et al. 1994, Jones et al. 2008). These promoters are described in the literature and are well-known to the person skilled in the art.

In one embodiment, the polynucleotides of the present invention are expressed in plants of the Solanaceae family, in particular in plants of the species Solanum tuberosum L., under the control of a strong constitutive promoter such as the 35S promoter. In another embodiment of the invention, the polynucleotides of the present invention are expressed in plants of the Solanaceae family and in particular in plants of the species Solanum tuberosum L. under the control of a specific root promoter.

In a preferred embodiment, the polynucleotides of the present invention are expressed under the control of the promoter of SEQ ID No. 1 and in particular under the control of the polynucleotide or of a polynucleotide fragment from position 1 to position 1657 of SEQ ID No. 1.

The expression cassettes of the present invention can further include any other sequence necessary for the expression of polypeptides or polynucleotides. Notably, any regulatory sequence that increases the expression level of the coding sequence inserted into the expression cassette can be used. According to the invention, in combination with the promoter regulatory sequence, other regulatory sequences located between the promoter and the coding sequence, such as transcription activators (enhancers), notably can be used.

A wide variety of termination sequences, which terminate mRNA transcription and polyadenylation, can be used in the expression cassettes of the invention. Any functional termination sequence in the selected host organism can be used.

For expression in plants of the Solanaceae family, expression cassettes comprising a terminator selected from the termination sequence of SEQ ID No. 1 (8811-10046) or termination sequences of ubiquitin genes, for example, will be chosen.

Advantageously, the expression cassettes of the present invention are inserted into a vector.

Vectors

The invention also relates to vectors comprising a polynucleotide of the invention or an expression cassette of the invention.

The present invention thus also relates to replication or expression vectors for transforming a host organism, comprising at least one polynucleotide or expression cassette of the present invention. This vector notably can be a plasmid, cosmid, bacteriophage, virus or artificial chromosome into which is inserted a polynucleotide or expression cassette of the invention. Techniques for constructing these vectors and inserting a polynucleotide of the invention into these vectors are well-known to the person skilled in the art.

Generally, any vector capable of surviving, self-replicating, propagating or becoming inserted into the genome of a host cell or host organism in order to induce notably the expression of a polynucleotide or a polypeptide can be used. The person skilled in the art will choose the appropriate vectors as a function of the host organism to be transformed and as a function of the transformation technique implemented.

Preferably, the vectors of the present invention enable the expression of a polynucleotide or a polypeptide of the invention in a plant of the Solanaceae family or in a plant cell, a seed or plant tissue from a plant of the Solanaceae family.

Among the vectors typically used to transform potato mention may be made notably of pBIN19 and derivatives (Bevan et al. 1984), the pCAMBIA series and derivatives, the pPZP series (Hajdukiewicz et al. 1994) and derivatives, in particular Gateway®-compatible vectors, for example p*GW (Karimi et al. 2002), the pGWB series (Nakagawa et al. 2009) or the vectors described in reviews by Hellens et al. (2000) and Karimi et al. (2007).

Host Cells and Organisms

The present invention also relates to a method for transforming a host cell or organism by integrating into said cell or said host organism at least one polynucleotide, expression cassette or vector of the invention. The polynucleotide can be integrated into the genome of the host cell/organism or can self-replicate in a stable manner in the host cell/organism. Methods for transforming host cells/organisms are well-known to the person skilled in the art and are widely described in the literature.

The invention thus also relates to a host cell transformed with a polynucleotide of the invention, an expression cassette of the invention or a vector of the invention. Preferably, these transformed cells are plant cells or plant cell protoplasts.

The present invention further relates to a host organism transformed with a polynucleotide, expression cassette or vector of the invention. In the context of the invention, the expression “host organism” refers in particular to any unicellular or multicellular, lower or higher organism. “Host organism” refers to a nonhuman organism. Preferably, the host organism transformed is a plant, a seed or plant tissue.

Thus, the invention also relates to a plant transformed with a polynucleotide of the invention, an expression cassette of the invention or a vector of the invention.

Preferably, the transformed plant is a plant of the Solanaceae family in which the GpaV gene is expressed or overexpressed in order to confer to this plant resistance to Globodera nematodes.

Advantageously, the transformed plant is selected from plants of the species Solanum tuberosum L., Solanum phureja, Solanum lycopersicum L., Solanum melongena L. and Nicotiana tabacum and plants of the genus Capsicum.

More preferentially, the transformed plant is potato and more particularly a commercially exploited potato cultivar.

Plants of the Solanaceae family can be rendered nematode-resistant by transformation with a polynucleotide, expression cassette or vector of the invention. The transformed plants are thus typically transgenic plants having integrated into their genome a polynucleotide, expression cassette or vector of the invention.

These cells, host organisms and transformed plants express a polynucleotide or polypeptide of the invention.

However, the invention also relates to plants, and particularly commercially cultivated plants of the Solanaceae family into which a polynucleotide of the present invention, notably the polynucleotide of SEQ ID No. 1, is introduced by introgression.

Thus, the invention relates to plants selected from the species Solanum tuberosum L., Solanum phureja, Solanum lycopersicum L., Solanum melongena L. and Nicotiana tabacum and plants of the genus Capsicum comprising a polynucleotide of the invention.

The invention also relates to plants selected from the species Solanum tuberosum L., Solanum phureja, Solanum lycopersicum L., Solanum melongena L. and Nicotiana tabacum and plants of the genus Capsicum comprising a polynucleotide of Solanum sparsipilum consisting of a genomic fragment of size smaller than 15 kbp, 20 kbp, 50 kbp, 200 kbp, 250 kbp, 500 kbp or 1 Mbp comprising a GpaV gene resistance allele of the invention, a polynucleotide of the present invention or more preferentially the polynucleotide of SEQ ID No. 1.

Preferentially, the invention relates to a potato (Solanum tuberosum or Solanum phureja) comprising a polynucleotide from Solanum sparsipilum consisting of a genomic fragment of size smaller than 15 kbp, 20 kbp, 50 kbp, 200 kbp, 250 kbp, 500 kbp or 1 Mbp comprising a GpaV gene resistance allele of the invention, a polynucleotide of the present invention or more preferentially the polynucleotide of SEQ ID No. 1.

Preferentially, the invention relates to a potato (Solanum tuberosum or Solanum phureja) comprising a polynucleotide of the invention and in particular a polynucleotide of SEQ ID No. 1.

More preferentially, the invention relates to a potato (Solanum tuberosum L. subsp. tuberosum) comprising a polynucleotide of the invention and in particular a polynucleotide of SEQ ID No. 1.

Methods for Conferring Nematode Resistance to Plants of the Solanaceae Family

The Solanum sparsipilum GpaV gene conferring nematode resistance was identified. This gene can now be introduced into Solanaceae species of interest.

To introduce the GpaV gene into plants of the Solanaceae family, it is essential to have available molecular markers specific to the GpaV gene of the present invention. These molecular markers are notably primers or probes derived from SEQ ID No. 1. These molecular markers in the form of primers or probes can notably be identified by aligning the polynucleotide of SEQ ID No. 1 coding for a GpaV gene resistance allele with the polynucleotides of SEQ ID Nos. 34-36 coding for sensitivity alleles of the GpaV nematode-resistance gene.

The invention thus also relates to selection markers derived from the polynucleotides of the present invention and to the use thereof for the marker-assisted selection of nematode-resistant plants and notably plants of the Solanaceae family such as potatoes. In a preferred embodiment of the invention, the pair of primers of SEQ ID Nos. 32-33 is used for the marker-assisted selection of nematode-resistant plants.

Another aspect of the invention is thus the use of polynucleotide primers or probes derived from SEQ ID No. 1 for detecting nematode-resistant plants of the Solanaceae family, for detecting plants expressing a polynucleotide of SEQ ID Nos. 1, 3, 5, 7, 9 or 11 or for detecting plants comprising the GpaV nematode resistance gene of SEQ ID No. 1.

The invention thus also relates to methods for detecting nematode-sensitive or -resistant plants of the Solanaceae family implementing the probes or primers derived from SEQ ID No. 1 of the present invention. Preferably, these probes or primers are fragments of at least 15 nucleotides of the polynucleotide of SEQ ID No. 1. In a preferred embodiment, the nematode-resistant plants of the Solanaceae family are detected with the pair of primers of SEQ ID Nos. 32-33.

This detection can be carried out according to methods well-known to the person skilled in the art such as PCR or hybridization.

Preferably, the GpaV gene is inserted into the genome of the plant of interest by transformation or transgenesis. The species of interest are notably plants selected from the species Solanum tuberosum L., Solanum phureja, Solanum lycopersicum L., Solanum melongena L. and Nicotiana tabacum and plants of the genus Capsicum.

The invention thus relates to a method for conferring Globodera nematode resistance to a plant of the Solanaceae family, comprising the following steps:
transforming the plant with a polynucleotide of the invention, an expression cassette of the invention or a vector of the invention;
selecting a plant resistant to Globodera nematodes.

Preferably, a nematode-resistant plant is selected using markers or primers derived from the polynucleotides of the present invention.

Transgenesis consists in introducing into the genome of the host plant a DNA fragment coding for a gene involved in the expression of a characteristic of interest. The most commonly used method is transformation via Agrobacterium tumefaciens. The DNA fragment is recombined in a binary vector (see description of vectors) which will be introduced into a strain of Agrobacterium tumefaciens by electroporation or heat shock. This bacterial strain will be used to infect plant tissues or protoplasts, and the DNA fragment of interest will be integrated into the genome of certain cells. These cells will be cultured on a medium that promotes regeneration, in order to regenerate a whole plant. The transformed plants will be selected either by culturing on a medium supplemented with an antibiotic or herbicide if a gene of resistance to the antibiotic or herbicide was transferred jointly with the gene of interest, or directly using molecular techniques such as PCR with specific primers for the gene of interest (Vetten et al., 2003). An alternative technique to transformation via Agrobacterium tumefaciens is transformation using biolistics. DNA containing the gene of interest is deposited on metal beads (in general of gold), and these metal beads are projected into plant tissue via a particle gun. The DNA penetrates the cells and in certain cases integrates into the genome. Transformed plants are selected in the same way as for transformation via Agrobacterium tumefaciens.

In another preferred embodiment, the GpaV nematode-resistance gene is inserted into the plant of interest by introgression. In this case, the plant of interest is preferably potato and the introgression comprises crossing a plant of the species Solanum tuberosum L. ssp. tuberosum, Solanum tuberosum L. ssp. andigena or Solanum phureja with a wild nematode-resistant plant related to potato, Solanum sparsipilum. These techniques of introgression followed by backcrossing to return to the genetic background of the recipient (plant of interest) are well-known to the person skilled in the art.

These introgression methods are made possible by identification of the GpaV gene and its polynucleotide sequence. The polynucleotides of the present invention and the markers or primers that can be derived from these polynucleotides can be used notably in marker-assisted selection.

The invention thus relates to a method for rendering resistant to Globodera nematodes a plant of the species Solanum tuberosum L. ssp. tuberosum, Solanum tuberosum L. ssp. andigena or Solanum phureja, comprising the following steps:
introgression of a segment of Solanum sparsipilum genomic DNA comprising a polynucleotide of the invention into a Solanum tuberosum L. subsp. tuberosum plant;
selecting, with molecular markers derived from SEQ ID No. 1, a Globodera nematode-resistant plant of the species Solanum tuberosum L. ssp. tuberosum, Solanum tuberosum L. ssp. andigena or Solanum phureja.

Finally, the Solanum sparsipilum genomic fragment comprising the polynucleotide of SEQ ID No. 1 introgressed into a plant of interest preferably has a size smaller than 15 kbp, 20 kbp, 50 kbp, 200 kbp, 250 kbp, 500 kbp, 1 Mbp or 2 Mbp.

Solanum sparsipilum is a diploid species whereas Solanum tuberosum is a tetraploid species. The introgression of a S. sparsipilum gene into the S. tuberosum genome thus requires a change of ploidy. Diploid S. tuberosum clones can be obtained by in situ parthenogenesis, or by the culture of anthers or ovules. These diploid clones are sexually compatible with S. sparsipilum. Conversely, diploidy can be changed to tetraploidy either by using the ability of certain wild or cultivated potato clones to produce diploid gametes, or by inducing polyploidization by culturing in vitro or treating with chemical agents such as colchicine. Introgression of a gene from a diploid wild species into the genome of a tetraploid cultivated species can thus be achieved by a succession of diploid or tetraploid pseudo-backcrosses (the S. tuberosum clone is changed at each crossing because potato poorly tolerates consanguinity). In each new generation, individuals with the gene of interest (or more exactly the resistance allele to the gene in question) are selected on the basis of markers (in the case of interest herein, but selection can also be made phenotypically). If the selection was made in terms of diploidy, it is necessary to return to tetraploidy after four to five generations in order to obtain a plant of good agronomic quality. If a S. sparsipilum diploid clone was doubled from the start, there is no subsequent change in ploidy but it is more difficult to eliminate unfavorable alleles (having influence on glycoalkaloid level and agronomic and gustatory quality) provided by the genome of the wild parent.

### FIGURES

FIG. 1: Genotype and phenotype of individuals with a recombination event between markers MS063_{—}2 and Z751_{—}2R.

### EXAMPLES

### I. Plant Material

The GpaVₛₚₗ QTL was mapped using an interspecific lineage (named 96D31/00D53) of 239 diploid clones from the crossing of the two parental accessions spl329.18 and Caspar H3 (Caromel et al. 2005). The G. pallida-resistant parent, spl329.18, is a clone diploid of the accession of S. sparsipilum PI310984, coming from the of Sturgeon Bay collection (USA). The sensitive parent, Caspar H3, is a dihaploid clone obtained by in situ parthenogenesis at the French National Institute for Agricultural Research (INRA; Ploudaniel, France) from the Caspar tetraploid variety. 1393 additional plants, from the same crossing, were analyzed for the high-resolution mapping of the GpaVₛₚₗ QTL.

The genotypes 96D31.75 and 96D31.69 belong to the 96D31 lineage. The 96D31.75 genotype has the sensitivity allele to the GpaVₛₚₗ QTL and has the resistance allele to the GpaXIₛₚₗ QTL. The 96D31.69 genotype has the sensitivity allele to the GpaVₛₚₗ QTL and the sensitivity allele to the GpaXIₛₚₗ QTL (Caromel et al. 2005).

Two genotypes of the 96D31 lineage, heterozygous for resistance to the GpaVₛₚₗ locus, were crossed to give rise to the 05D2 lineage. Forty individuals of this lineage were analyzed with markers flanking the GpaVₛₚₗ QTL in order to identify the 05D2.12 genotype, homozygous at the GpaVₛₚₗ locus.

### II. Tests of G. pallida Resistance for Genetic Mapping

The resistance of potato clones to G. pallida, for the mapping of the GpaVₛₚₗ QTL and the high-resolution mapping of the gene underlying the GpaVₛₚₗ QTL, was evaluated by counting neoformed cysts as described in the publication by Caromel et al. (2005). Briefly, four tubers per genotype were planted separately in plastic pots filled with 400 cm³ of a mixture of sand and loam. Ten G. pallida cysts, Pa2/3 pathotype (Chavornay population), were added to each pot in order to obtain an infestation density of 5 to 10 G. pallida juveniles per gram of soil. The plants were cultivated in a greenhouse for four months. The sensitive control was the Desiree variety. Neoformed cysts were separated from the substrate by elutriation. They were counted separately for each pot. The raw data were transformed by a logarithmic function: log 10 (number of cysts+1).

### III. Development of Novel Markers and Genetic Mapping of the Gene Underlying the GpaVₛₚₗ QTL

The GpaVₛₚₗ QTL was mapped on chromosome V of spl329.18, in an interval of 5 cM between the GP21-SCAR and TG432P-CAPS markers (Caromel et al. 2005). In order to develop novel molecular markers in this interval, an analysis of homology was carried out between the GP21-SCAR and TG432P-CAPS sequences and the sequences of Solanum demissum BAC clones available in public databases. The sequence of the TG432P-CAPS marker made it possible to identify the AC150162 BAC clone, which overlaps the AC151803 and AC154033 BAC clones (Kuang et al. 2005). The ASC231, ASC240, Z751F_{—}2R, ASC102 and MS092 markers were developed from the sequences of the AC151803 and AC154033 BACs. The MS063_{—}2 marker was developed from the expressed sequence tag (EST) of the CK864217 potato. The primer sequences for amplifying these markers are indicated in the sequence listing.

In order to map the gene underlying the GpaVₛₚₗ QTL as a marker locus, the quantitative resistance data (number of neoformed G. pallida cysts) were converted into qualitative data (resistant vs. sensitive). A clone was regarded as having the resistance allele to the GpaVₛₚₗ QTL when less than 13 neoformed cysts on average per pot were found, and a clone was regarded as having the sensitivity allele to the GpaVₛₚₗ QTL when more than 70 neoformed cysts on average per pot were found. The clones on which between 13 and 70 neoformed cysts on average per pot were found were not taken into account. In this way, the gene underlying the GpaVₛₚₗ QTL was mapped, in the mapping lineage of 239 clones used to detect the QTL (Caromel et al. 2005), between the MS063_{—}2 and Z751F_{—}2R markers, at 0.4 cM from each of these two markers.

In order to find recombination events closer to the gene underlying the GpaVₛₚₗ QTL, the 1393 clones, from crossing of the same spl329.18 and Caspar H3 parents, were sorted using molecular markers in several steps. A first sorting was carried out on all of these plants with GP21-SCAR (Caromel et al. 2005) and GP179 markers (Meksem et al. 1995) widely flanking the confidence interval of the GpaVₛₚₗ QTL but easy to use on large numbers of samples. The 107 clones with a recombination event between these two markers were then genotyped with the MS063_{—}2 and Z751F_{—}2R markers, which made it possible to identify 12 clones with a recombination event between these two markers.

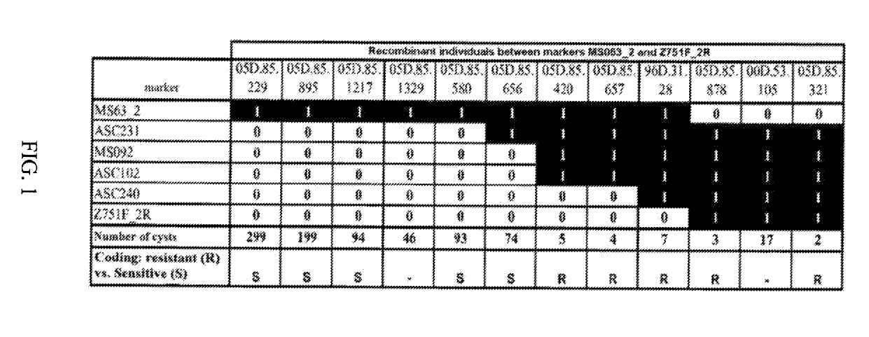

The 12 clones with a recombination event between MS063_{—}2 and Z751F_{—}2R were phenotyped according to the protocol described above for mapping the QTL, with four repetitions per clone. On the 12 clones tested, five were regarded as resistant, five as sensitive and two were not taken into account (FIG. 1). The 12 clones with a recombination event between MS063_{—}2 and Z751F_{—}2R were also genotyped with five markers defined according to the sequence of the S. demissum BAC clones (AC151803 and AC154033): markers ASC231, ASC240, 2751, ASC102 and MS092. Mapping the resistance characteristic and these five novel markers made it possible to locate the gene underlying the GpaVₛₚₗ QTL between the ASC231 and ASC240 markers (FIG. 1). One recombination event separates the GpaVₛₚₗ locus from the ASC231 marker and two recombination events separate it from the ASC240 marker. No recombination event made it possible to separate the ASC102 and MS092 markers from G. pallida resistance.

### IV. Chromosome Landing with the GpaVₛₚₗ Locus and Identification of Candidate Genes

On the sequence of the AC151803 S. demissum BAC clone, the ASC231 and ASC240 markers are at a distance of 30 kbp. Three putative genes were annotated in these 30 kbp:
SDM1_{—}55t00002: leucine-rich repeat family protein (position 18476 to 20167),
SDM1_{—}55t00003: disease resistance protein, putative (position 22149 to 26161),
SDM1_{—}55t00004: putative mTERF domain containing protein, identical (position 28630 to 29641).

Several resistance genes, described in the bibliography (van Ooijen et al. 2007), comprise a Toll/interleukin-1 receptor (TIR) homology domain, an NB-ARC (nucleotide-binding adapter shared by APAF-1 resistance proteins, and CED-4) domain and a leucine-rich repeat (LRR) domain. The TIR and NB-ARC domains are detected in the SDM1_{—}55t00003 putative gene and the LRR domain is detected in the SDM1_{—}55t00002 putative gene. These two putative genes, annotated on the sequence of the S. demissum BAC clone, a species sensitive to G. pallida, could thus form a single functional gene in the accession of G. pallida-resistant S. sparsipilum. Another argument in this direction comes from the sequence of the Bs4 gene conferring resistance to Xanthomonas campestris pv. tomato in tomato (Schornack et al. 2004). This gene, mapped in a collinear position at the GpaVₛₚₗ locus, has three domains, namely TIR, NBS and LRR. It was thus probable that the two putative genes SDM1_{—}55t00002 and SDM1_{—}55t00003 in reality only formed one in S. sparsipilum.

In order to verify this hypothesis, cDNA corresponding to this locus in S. sparsipilum were amplified and sequenced. Sequences corresponding to the TIR, NB-ARC and LRR domains are found on same cDNA (sequence cDNA_H1), thus confirming that the two putative genes detected on the S. demissum sequence actually form only one in S. sparsipilum. Since genes of the TIR-NB-ARC-LRR family are classic resistance genes, it was decided to clone the resistance allele of this gene in spl329.18 to validate functionally its involvement in G. pallida resistance.

### V. Obtaining the Sequence of the Resistance Allele of the TIR-NB-ARC-LRR Gene Underlying the GpaVₛₚₗ QTL

The Z1505_{—}6F/Z1505_R primer pair was defined from sequences of overlapping BAC clones of S. demissum (AC151803 and AC154033) and of S. lycopersicum (AC232763), to amplify by PCR the totality of the coding sequence of the TIR-NB-ARC-LRR gene and roughly 2000 base pairs of 5′ and 3′ flanking sequences. Amplification was carried out in 50 μl from 50 ng of DNA of the 05D2.12 genotype, from a brother-sister cross between two genotypes of the mapping lineage and homozygote at the GpaVₛₚₗ locus, using a unit of TaKaRa Ex Taq HS (Lonza, Verviers, Belgium) according to the condition described in the protocol provided by the supplier, with the following amplification program: an initial denaturation step at 94° C. for 2 minutes, followed by 40 cycles comprising a denaturation step at 98° C. for 10 seconds and a primer hybridization and complementary strand synthesis step at 68° C. for 10 minutes, followed by a final elongation step at 72° C. for 15 minutes. The size and quantity of the PCR product obtained were estimated by migrating 2 μl of PCR product on a 0.8% agarose gel. Eight independent amplifications were carried out. The PCR products were purified by precipitation with two volumes of absolute ethanol and sodium acetate at 0.3 M final concentration, washed twice with 600 μl of 70% ethanol and resuspended in ultrapure water. The purified PCR product was sent to be sequenced at Cogenics (Meylan, France). The sequence obtained was used as the reference sequence for the subsequent amplification and cloning step.

### VI. Functional Validation of the TIR-NB-ARC-LRR Gene Underlying the GpaVₛₚₗ QTL

The Z1505_{—}8F/Z1505_{—}4R primer pair was defined from the sequence of the fragment obtained by PCR with the Z1505_{—}6F/Z1505_R primer pair on DNA of the 05D2.12 genotype. These primers were used to amplify by PCR the totality of the TIR-NB-ARC-LRR gene with 1821 base pairs of sequences upstream (before ATG) and 1720 base pairs of sequences downstream after the stop codon, from DNA of the G. pallida-resistant spl329.18 genotype. Amplification was carried out 8 times 20 μl using Herculase II Fusion Enzyme (Agilent Technologies, Massy, France) according to the supplier's instructions. The following amplification program was used: an initial denaturation step at 94° C. for 2 minutes, followed by 20 cycles comprising a denaturation step at 98° C. for 10 seconds and a primer hybridization and complementary strand synthesis step at 68° C. for 5 minutes, followed by a final elongation step at 72° C. for 15 minutes.

After verification on a 0.8% agarose gel, each amplification product was cloned separately in the pBIN19 binary vector (Bevan et al. 1984) digested by the SalI enzyme, using the Clontech In-Fusion 2.0 Dry-Down PCR Cloning Kit (resold by Ozyme, Saint-Quentin-en-Yvelines, France) according to the supplier's instructions. The product of each reaction was used to transform 50 μl of the NEB 10-beta Competent E. coli strain, and then cultured according to the supplier's instructions (New England BioLabs, resold by Ozyme, Saint-Quentin-en-Yvelines, France). For each of the eight independent clonings, 12 isolated white colonies (the color indicating the presence of an insert in the plasmid (Bevan et al. 1984)) were cultured in 2 ml of LB medium containing 50 μg kanamycin per ml. An aliquot of the culture was used to verify by PCR the presence of an insert of the expected size using the Z1505_{—}8F/Z1505_{—}4R primer pair. A positive clone by independent cloning was sent to be sequenced at Cogenics after purification of the plasmid with the kit Macherey-Nagel NucleoSpin Plasmid kit (Düren, Germany). Two of the eight clones sequenced had a sequence 100% identical to the reference sequence obtained from the PCR product. One of these two clones, GpaVₛₚₗ_{_{—}}clone8, was used for subsequent functional validation steps. The sequence of the cloned fragment corresponds to the “GpaVₛₚₗ genome sequence” of SEQ ID No. 1.

Two microliters of plasmid purified from GpaVₛₚₗ_{_{—}}clone8 was introduced by electroporation into the C58 Agrobacterium tumefaciens strain carrying the pGV2660 helper plasmid (Deblaere et al. 1985), using a standard procedure. The strain was cultured at 28° C. for roughly 24 hours in LB medium+kanamycin (50 μg/ml) until an optical density of 0.3 at 600 nm is obtained. Internode fragments (0.5-0.8 cm in length), from cuttings of potato cultivated in vitro, were incubated for 20 minutes in 15 ml of bacterial solution. The internode fragments were then dried on filter paper, co-cultured on “potato” co-culture medium (Table 1) supplemented with 0.9 mg/l of thiamin and 39 mg/l of acetosyringone and then placed in the dark for two days at 24° C.

TABLE 1
Composition of the “potato” medium

| | Quantity for 1 | |
|---|---|---|
| | liter of medium | |
| KNO₃ | 2.69 | g |
| NH₄NO₃ | 536 | mg |
| Ca(NO₃)₂, 4H₂O | 472 | mg |
| MgSO₄, 7H₂O | 418.6 | mg |
| KH₂PO₄ | 274 | mg |
| KCl | 350 | mg |
| H₃BO₃ | 6.2 | mg |
| MnSO₄, H₂O | 16.9 | mg |
| ZnSO₄, 7H₂O | 10.6 | mg |
| KI | 0.83 | mg |
| CuSO₄, 5H₂O | 0.025 | mg |
| CoCl₂, 6H₂O | 0.025 | mg |
| Na₂MoO₄, 2H₂O | 0.25 | mg |
| Myo-inositol | 100 | mg |
| Glycine | 2 | mg |
| Thiamine HCL | 0.5 | mg |
| Pyridoxine HCL | 0.5 | mg |
| Nicotinic acid | 0.5 | mg |
| SO₄Fe, 7H₂O | 37.3 | mg |
| Na₂EDTA | 27.8 | mg |
| Sucrose | 25 | g |
| Vitro Agar | 6 | g |

pH adjusted to 5.8 with KOH

After this period of co-culture, the explants were transferred to petri dishes on fresh “potato” medium rich in selection antibiotic (300 mg/l kanamycin), an antibiotic to eliminate bacteria (225 mg/l Timentin) and hormones promoting regeneration (0.1 mg/l ANA, 0.1 mg/l GA3, 1 mg/l BAP), and then placed in a culture chamber at 20° C. The culture medium was changed every 15 days. After culturing for two to three months, the regenerated plants were isolated from the explants and then transferred for rooting in culture tubes containing “potato” medium supplemented with 225 mg/l of Timentin and 300 mg/l of kanamycin. Plants arising from a transformation event and producing a root system on the kanamycin-supplemented medium were indexed and multiplied in order to have available a sufficient number of cuttings to carry out G. pallida resistance testing.

The G. pallida resistance of the transgenic plants was evaluated by an in vitro test. Six independent transformation events, regenerated from the 96D31.75 genotype having the sensitivity allele to the GpaVₛₚₗ QTL and the resistance allele to the QTL GpaXIₛₚₗ, were tested. These independent transformation events were named 96D31.75_A, 96D31.75_B, 96D31.75_C, 96D31.75_E, 96D31.75_F, and 96D31.75_G. The 96D31.75 genotype, transformed with the GUS reporter gene, was used as a control (named 96D31.75_GUS). Rooted cuttings were transferred to petri dishes containing “potato” medium without sucrose and in which Vitro Agar was replaced by 5 g/l Gelrite (Kalys, Saint Ismier, France). The dishes were placed vertically in a phytotron set at a temperature of 17° C., hygrometry 70%, 16-hour day length and 8-hour night, lighting intensity 250 μmol·photon·m²·s⁻¹. After two days, thirty roots were inoculated per transformation event and for the control (two roots per cutting and 15 cuttings per transformation event or for the control). Each root was inoculated with five G. pallida juveniles (Chavornay population) in the J2 stage and dishes containing the inoculated cuttings were placed horizontally in the phytotron. The inoculated roots were excised four weeks after inoculation, immersed for 5 minutes in 1% calcium hypochlorite solution and stained for 15 seconds in a solution of acid fuchsin (0.1% in 30% acetic acid) at 100° C. Nematodes are thus stained in red. The roots were then crushed between the slide and the cover glass, and nematodes in the various stages of development were counted under a microscope (400× magnification). They were divided into two categories: female stage and other (male, J2, J3) stages.

The data obtained for the six transformation events were compared to those obtained for the 96D31.75_GUS genotype control using a chi-squared (χ²) test with 1 degree of freedom. According to the χ² test, the transformation events 96D31.75_A, B, C, E and G are significantly different than the control used: they enabled the development of significantly fewer females than in the control (Table 2).

TABLE 2
Percentage of nematodes that developed into
females, 21 days after inoculation, in the roots of
plants transformed with the TIR-NBS-LRR gene. The
significance of the differences between the 96D31.75_A
to G transformants and the 96D31.75 control transformed
with the GUS gene was evaluated by χ² with 1 degree of
freedom.

| | Sample | % | | | |
|---|---|---|---|---|---|
| Event | size | females | χ² | Probability | Significance |
| 96D31.75_A | 116 | 21.6 | 3.94 | 0.04709 | The difference |
| | | | | | is significant |
| 96D31.75_B | 101 | 15.8 | 8.41 | 0.00374 | The difference |
| | | | | | is significant |
| 96D31.75_C | 72 | 11.1 | 11.45 | 0.00071 | The difference |
| | | | | | is significant |
| 96D31.75_E | 73 | 15.1 | 7.54 | 0.00604 | The difference |
| | | | | | is significant |
| 96D31.75_F | 90 | 26.7 | 1.15 | 0.28366 | The difference |
| | | | | | is significant |
| 96D31.75_G | 87 | 8.0 | 17.66 | 0.00003 | The difference |
| | | | | | is significant |
| Control | 85 | 34.1 | — | — | — |

The experiment was repeated a second time for the 96D31.75_C, E and G events and once again showed highly significant differences between these three transformants and the control (96D31.75_C: χ²=18.58, p=4.66×10⁻⁵; 96D31.75_E: χ²=36.70, p=1.36×10⁻⁹; 96D31.75_G: χ²=34.77, p=3.70×10⁻⁹). These experiments demonstrate that the TIR-NB-ARC-LRR gene is involved in G. pallida resistance.

In order to validate these results, the 96D31.69 genotype, possessing sensitivity alleles to both GpaVₛₚₗ and GpaXIₛₚₗ QTLs, was transformed with the same bacterial strain (GpaVₛₚₗ_{_{—}}clone8 bacterial clone, in the C58 Agrobacterium tumefaciens strain carrying the pGV2660 helper plasmid). Three independent transformation events (09D803.5, 09D.817.36 and 09D.817.42) were tested for their G. pallida resistance. The 96D31.69 genotype, transformed with the GFP reporter gene was used as a control. The resistance test was carried out as described above. Two roots from 5 to 15 cuttings were inoculated for each independent transformation event.

The data obtained for the three transformation events from the 96D31.69 genotype were compared to those obtained for the 96D31.69_GFP genotype control, using a χ² test with 1 degree of freedom. According to the χ² test, the three transformation events are significantly different than the control used: they enabled the development of significantly fewer females than in the control (Table 3).

TABLE 3
Percentage of nematodes that developed into
females, 28 days after inoculation, in the roots of the
plants from the 96D31.69 genotype, transformed with the
TIR-NBS-LRR gene.
The significance of the differences between the
09D803.5, 09D817.36, 09D817.42 transformants and the
96D31.69 control, transformed with the GFP reporter
gene, was evaluated using χ2 with 1 degree of freedom.

| | Sample | % | | | |
|---|---|---|---|---|---|
| Event | size | females | χ² | Probability | Significance |
| 09D803.5 | 56 | 16.0 | 7.45 | 0.00636 | The |
| | | | | | difference |
| | | | | | is significant |
| 09D817.36 | 65 | 16.9 | 7.42 | 0.00644 | The |
| | | | | | difference |
| | | | | | is significant |
| 09D817.42 | 44 | 11.4 | 9.70 | 0.00185 | The |
| | | | | | difference |
| | | | | | is significant |
| 96D31.69_GFP | 36 | 41.7 | — | — | — |
| control | | | | | |

### VII. Determination of the Coding Sequence of the TIR-NB-ARC-LRR Gene Underlying the GpaVₛₚₗ QTL

The coding sequence of the gene was identified from RNA from tissue fragments taken from the 05D2.12 genotype. Tissue fragments were taken from roots inoculated and not inoculated by G. pallida at 6 hours, 2 days and 4 days post-inoculation, as well as from leaves, seeds and stems. The RNA from each sample was extracted with Qiagen's RNeasy Plant Mini Kit (Courtaboeuf, France) according to the supplier's instructions and then assayed by spectrophotometry. The RNA of the various samples was mixed in equivalent proportions. One microgram of total RNA was reverse-transcribed with 200 units of SuperScript™ II Reverse Transcriptase (Invitrogen, Cergy Pontoise, France), according to the supplier's instructions.

The ends of the RNA transcribed from the TIR-NB-ARC-LRR gene were determined by 5′ and 3′ RACE using the SMART RACE cDNA Amplification Kit (Clontech, resold by Ozyme). A series of RT-PCR experiments were carried out using primers defined for the RACE experiments and primers defined on the 5′ and 3′ ends of the cDNA. The cDNA fragments were amplified by PCR with one unit of TaKaRa Ex Taq HS (Lonza, Verviers, Belgium) under the condition described in the protocol provided by the supplier, with the following amplification program: an initial denaturation step at 94° C. for 2 minutes, followed by 40 cycles comprising a denaturation step at 98° C. for 10 seconds, a primer hybridization step at 60° C. for 20 seconds and a complementary strand synthesis step at 72° C. for 3 minutes, and followed by a final elongation step at 72° C. for 10 minutes. The amplified products were cloned and/or purified and sent to be sequenced at Cogenics. The sequences obtained following the RACE and RT-PCR experiments were aligned with the sequence of the genomic DNA using the SIM4 (http://pbil.univ-lyonl.fr/sim4.php) and Multalin software (http://bioinfo.genotoul.fr/multalin/multalin.html).
The alignments made it possible to determine the position of introns and exons and demonstrated an important use of alternative splicing mechanisms during mRNA maturation.

After the promoter region, the GpaVₛₚₗ gene comprises five exons and four introns. Exon 1 comprises the 5′ untranslated region (5′ UTR) and the TIR domain, exon 2 comprises the NB-ARC domain, exon 3 comprises the start of the LRR domain, exon 4 comprises the end of the LRR domain up to the stop codon and the beginning of the 3′ untranslated region (3′ UTR), and exon 5 comprises the end of the 3′ UTR. mRNA corresponding to various alternative splicing mechanisms were observed: retention of intron 1, intron 2 and/or intron 3, skipping of exon 3, use of a cryptic acceptor site in exon 2 leading to a deletion of the first 966 base pairs of exon 2. Alternative polyadenylation sites were identified in exons 2, 3 and 5 as well as in intron 3. Four transcription initiation sites were identified in exon 1. The retention of an intron led systematically to the appearance of a premature stop codon before the following exon. The nucleotide sequences, from ATG to the stop codon and resulting from the various possible types of splicing, correspond to the sequences cDNA_{—}1 to cDNA_{—}11.

### VIII. Evaluation of the Expression of the TIR-NB-ARC-LRR Gene Underlying the GpaVₛₚₗ QTL

The level of expression of the TIR-NB-ARC-LRR gene was determined for two conditions (I: inoculated with nematode; NI: not inoculated) and at three points in time (6 hours, 2 days and 4 days after parasitic infection) in different genotypes of the population from the cross between Caspar H3 and spl3219.18. The genotypes were selected according to their allelic combinations at both GpaVₛₚₗ and GpaXIₛₚₗ QTLs (Caromel et al. 2005). The 96D31.139 genotype possesses the resistance alleles to two QTLs (R5R11), the 96D31.03 genotype has the resistance allele to the GpaVₛₚₗ QTL and the sensitivity allele to the GpaXIₛₚₗ QTL (R5S11), and the 96D31.152 genotype possesses the sensitivity alleles to two QTLs (S5S11).

RNA was extracted from five 5 mm root fragments around the inoculation point or from the corresponding zone for the uninoculated plants using Qiagen's RNeasy Plant Mini Kit (Courtaboeuf, France). For each sample, 1 μg of RNA was reverse-transcribed with 200 units of SuperScript™ II Reverse Transcriptase (Invitrogen, Cergy Pontoise, France), according to the supplier's instructions. The reaction was then diluted 20 times with ultrapure water.

The expression level of the TIR-NB-ARC-LRR gene was measured by Q-RT-PCR with the SYBR Premix Ex Taq kit (TaKaRa) on an Mx3005® device (Stratagene) according to the supplier's instructions, using Q63 primers and the following program: an initial denaturation step at 95° C. for 2 minutes, followed by 40 cycles comprising a denaturation step at 95° C. for 20 seconds, a primer hybridization step at 55° C. for 20 seconds and a complementary strand synthesis step at 72° C. for 30 seconds. The denaturation curve for the amplified product was calculated after the following three steps: 95° C. for 1 minute, 55° C. for 30 seconds and 95° C. for 1 minute. Expression data for the cytosolic phosphoglycerate kinase (PGK) F gene was used as a reference to normalize the results obtained between the various samples (Coker and Davies 2003). It was amplified according to the same protocol, except for the primer hybridization temperature which was 50° C.

Statistical analyses for interpreting the raw data provided by the Mx3005P® QPCR System were carried out using the REST® software (Pfaffl et al. 2002), which functions on the basis of Ct values and Q-RT-PCR efficiency. A ratio is calculated of the relative expression between a target gene and a reference gene (or housekeeping gene) whose expression remains unchanged in the cell over time. This eliminates possible differences in the starting quantity of cDNA. If from one sample to the next the quantity of cDNA is greatly different, the housekeeping gene makes it possible to normalize the results.

This ratio is determined according to the formula:

$$R = \frac{{( {E}_{target} )}^{Δ CPtarget ( MEAN control - MEAN sample )}}{{( {E}_{ref} )}^{Δ CPref ( MEAN control - MEAN sample )}}$$

In the present case:
E_{target}=PCR efficiency with the target gene (TIR-NB-ARC-LRR).
MEAN control_{target}=Ct of the target gene in the control sample (for example, SS NI 6 h).
MEAN sample_{target}=Ct of the target gene in the unknown sample (for example, SS I 6 h).
E_{ref}=PCR efficiency with the PGK housekeeping gene.
MEAN control_{ref}=Ct of the PGK gene in the control sample (for example, SS NI 6 h).
MEAN sample_{ref}=Ct of the PGK gene in the unknown sample (for example, SS I 6 h).

The TIR-NB-ARC-LRR gene is expressed to a much greater degree in an R5R11 genotype than in an S5S11 genotype, at 6 hours (31 times more) and at 4 days (14 times more). Similarly, this gene is expressed 6 times greater in an R5R11 genotype than in an R5S11 genotype, 6 hours after nematode inoculation.

In the case of uninoculated plants, the TIR-NBS-LRR gene is also expressed to a greater extent, regardless of the kinetics, in an R5R11 genotype compared to an S5S11 genotype: 6 hours after infection it is expressed 16 times greater, 2 days later it is 13 times greater and after 4 days it is 234 times greater.

Differences in expression levels observed between genotypes possessing the sensitivity allele to the TIR-NBS-LRR gene (S5S11) and genotypes possessing the resistance allele (R5S11 and R5R11) suggest an influence of the gene promoter on expression of resistance. For genotypes having the resistance allele to the TIR-NBS-LRR gene, differences in expression levels observed for this gene between genotypes possessing sensitivity (R5S11) and resistance (R5R11) alleles to the weak-effect GpaXIₛₚₗ QTL also suggest that interaction between the two QTLs is related to an increase in the transcription level of the TIR-NB-ARC-LRR gene by the resistance allele to the GpaXIₛₚₗ QTL. It is likely that this increase in expression level of the TIR-NB-ARC-LRR gene occurs by the recognition (direct or indirect) of a specific motif of its promoter by the resistance allele to the QTL GpaXIₛₚₗ.

### IX. Identification of Sequences of Sensitivity Alleles from Two Accessions of S. sparsipilum and S. tuberosum

The Z1505_{—}8F/Z1505_{—}5R primer pair was used to amplify the GpaV locus from two accessions of S. sparsipilum (spl329.18 and spl504.5) and two diploid accessions of S. tuberosum (Caspar H3 and Rosa H1). The amplified products were cloned as described above. At least three clones per allele were sequenced. In the two accessions of S. sparsipilum spl329.18 and spl504.5, the resistance allele is identical and corresponds to the sequence SEQ ID No. 1. The sensitivity alleles from these two accessions are different and correspond to sequences SEQ ID No. 34 and SEQ ID No. 35. A single allele was cloned from S. tuberosum; this allele is identical in the two G. pallida-sensitive accessions, Caspar H3 and Rosa H1. The sequence of this allele corresponds to the sequence SEQ ID No. 36.

### X. Definition of a Marker for Following the GpaVₛₚₗ Resistance Allele During the Selection Process

S. sparsipilum clones spl329.18 and spl504.5 are heterozygous at the GpaV locus. The resistance allele, GpaVₛₚₗ, is identical in these two clones. On the other hand, the sensitivity alleles of these two clones are different. The S. tuberosum sensitivity allele, isolated from the Caspar H3 and Rosa H1 clones, is different from all the alleles isolated in S. sparsipilum. The genomic sequences of the GpaVₛₚₗ resistance allele and the three sensitivity alleles were aligned with the Multalin software (http://multalin.toulouse.inra.fr/multalin/multalin.html). Regions polymorphic between the resistance allele and the sensitivity alleles were identified. PCR primers flanking these regions were defined in order to amplify these polymorphic regions for all the alleles. The specificity of the primers was verified by a search for homology (blastn) with all the potato genomic sequences available in public databases: only primer pairs with strong homology with chromosome 5 BAC clone sequences alone were retained. The 23461 primer pair was defined to amplify a fragment of 1050 to 1100 base pairs depending on the allele, comprising part of exon 3 and part of intron 3.

The primers for amplifying this marker are as follows:

| | |
|---|---|
| Z3461F: | GACCATGACAGTGGAAGCAA |
| Z3461R: | TGGTTGTCAGAAGCAAATGAAG |

Digestion of the amplification products with the MboI restriction enzyme produces three fragments of 208, 229 and 622 base pairs for the GpaVₛₚₗ resistance allele and only two fragments for the three sensitivity alleles (one fragment of 229 bp and one fragment ranging between 845 bp and 856 bp depending on the sensitivity allele). The various fragments are visualized after electrophoresis of the MboI-digested amplification products in a 2% agarose gel. This marker makes it possible to specifically distinguish the GpaVₛₚₗ resistance allele from sensitivity alleles, whether from S. tuberosum or S. sparsipilum.

### REFERENCES

Bevan, M. 1984. A New Agrobacterium Vector for Plant Transformation. Heredity 53:577-578.
Bryan, G. J., McLean, K., Bradshaw, J. E., Jong, W. S. d., Phillips, M., Castelli, L. and Waugh, R. 2002. Mapping QTLs for resistance to the cyst nematode Globodera pallida derived from the wild potato species Solanum vernei. Theor. Appl. Genet. 105:68-77.
Caromel, B., Mugniery, D., Lefebvre, V., Andrzejewski, S., Ellisseche, D., Kerlan, M. C., Rousselle, P. and Rousselle-Bourgeois, F. 2003. Mapping QTLs for resistance against Globodera pallida (Stone) Pa2/3 in a diploid potato progeny originating from Solanum spegazzinii. Theor. Appl. Genet. 106:1517-1523.
Caromel, B. 2004. [In French] Cartographie génétique et étude de QTL conférant la résistance au nématode à kyste Globodera pallida (Stone) chez la pomme de terre (Solanum tuberosum ssp. tuberosum L.). PhD Thesis, Paris XI University, France.
Caromel, B., Mugniéry, D., Kerlan, M. C., Andrzejewski, S., Palloix, A., Ellissèche, D., Rousselle-Bourgeois, F. and Lefebvre, V. 2005. Resistance quantitative trait loci originating from Solanum sparsipilum act independently on the sex ratio of Globodera pallida and together for developing a necrotic reaction. Mol. Plant-Microbe Interact. 18:1186-1194.
Coker, J. S, and Davies, E. 2003. Selection of candidate housekeeping controls in tomato plants using EST data. BioTechniques 35:740-748.
Deblaere, R., Bytebier, B., De Greve, H., Deboeck, F., Schell, J., Van Montagu, M. and Leemans, J. 1985. Efficient octopine Ti-plasmid derived for Agrobacterium-mediated gene transfer. Nucl. Acids. Res. 13:4777-4788.
de Vetten, N., Wolters, A. M., Raemakers, K., van der Meer, L, ter Stege, R., Heeres, E., Heeres, P. and Visser, R. 2003. A transformation method for obtaining marker-free plants of a cross-pollinating and vegetatively propagated crop. Nat. Biotechnol. 21:439-442.
Garbarino, J. E. and Belknap, W. R. 1994. Isolation of a Ubiquitin-Ribosomal Protein Gene (Ubi3) from Potato and Expression of Its Promoter in Transgenic Plants. Plant mol. Biol. 24: 119-127.
Garbarino, J. E., Oosumi, T. and Belknap, W. R. 1995. Isolation of a Polyubiquitin Promoter and Its Expression in Transgenic Potato Plants. Plant Physiol. 109:1371-1378.
Hajdukiewicz, P., Svab, Z. and Maliga, P. 1994. The small, versatile pPZP family of Agrobacterium binary vectors for plant transformation. Plant mol. Biol. 25:989-994.
Hellens, R., Mullineaux, P. and Klee, H. 2000. A guide to Agrobacterium binary Ti vectors. Trends Plant Sci. 5:446-451.
Jones, M. O., Manning, K., Andrews, J., Wright, C, Taylor, I. B. and Thompson, A. J. 2008. The promoter from SIREO, a highly-expressed, root-specific Solanum lycopersicum gene, directs expression to cortex of mature roots. Functional Plant Biology 35:1224-1233.
Karimi, M., Inze, D. and Depicker, A. 2002. Gateway™ vectors for Agrobacterium-mediated plant transformation. Trends Plant Sci. 7:193-195.
Karimi, M., Bleys, A., Vanderhaeghen, R. and Hilson, P. 2007. Building blocks for plant gene assembly. Plant Physiol. 145:1183-1191.
Kort, J., Ross, H., Rumpenhorst, J. H. and Stone, A. R. 1977. An international scheme for classifying pathotypes of potato cyst nematodes Globodera rostochiensis and G. pallida. Nematologica 23:333-339.
Kuang, H. H., Wei, F. S., Marano, M. R., Wirtz, U., Wang, X. X., Liu, J., Shum, W. P., Zaborsky, J., Talion, L. J., Rensink, W., Lobst, S., Zhang, P. F., Tornqvist, C. E., Tek, A., Bamberg, J., Helgeson, J., Fry, W., You, F., Luo, M. C., Jiang, J. M., Buell, C. R. and Baker, B. 2005. The R1 resistance gene cluster contains three groups of independently evolving, type I R1 homologues and shows substantial structural variation among haplotypes of Solanum demissum. Plant Journal 44:37-51.
Meksem, K., Leister, D., Peleman, J., Zabeau, M., Salamini, F. and Gebhardt, C. 1995. A high-resolution map of the vicinity of the R1 locus on chromosome V of potato based on RFLP and AFLP markers. Mol. gen. Genet. 249:74-81.
Mugniéry, D., Fouville, D., Dantec, J. P., Pellé, R., Rousselle-Bourgeois, F. and Ellissèche, D. 2001. [In French] Résistance à Globodera pallida Pa2/3 chez Solanum sparsipilum. Nematology 3:619-626.
Nakagawa, T., Ishiguro, S. and Kimura, T. 2009. Gateway vectors for plant transformation. Plant Biotechnology 26:275-284.
Opperman, C H., Taylor, C. G. and Conkling, M. A. 1994. Root-Knot Nematode-Directed Expression of a Plant Root-Specific Gene. Science 263:221-223.
Pfaffl, M. W., Horgan, G. W. and Dempfle, L. 2002. Relative expression software tool (REST (c)) for group-wise comparison and statistical analysis of relative expression results in real-time PCR. Nucl. Acids. Res. 30(9): e36.
Rouppe van der Voort, J., Wolters, P., Folkerstma, R., Hutten, R., van Zanvoort, P., Vinke, H., Kanyuka, K., Bendahmane, A., Jacobsen, E., Janssen, R. and Bakker, J. 1997. Mapping of the cyst nematode resistance locus Gpa2 in potato using a strategy based on co-migrating AFLP markers. Theor. Appl. Genet. 95:874-880.
Rouppe van der Voort, J. N. A. M., Lindeman, W., Folkertsma, R., Hutten, R. C. B., Overmars, H., Van der Vossen, E., Jacobsen, E. and Bakker, J. 1998. A QTL for broad-spectrum resistance to cyst nematode species (Globodera ssp.) maps to a resistance gene cluster in potato. Theor. Appl. Genet. 96:654-661.
Rouppe van der Voort, J. N. A. M., van der Vossen, E., Bakker, E., Overmars, H., van Zandvoort, P., Hutten, R., Klein Lankhorst, R. and Bakker, J. 2000. Two additive QTLs conferring broad-spectrum resistance in potato to Globodera pallida are localized on resistance gene clusters. Theor. Appl. Genet. 101:1122-1130.
Schornack, S., Ballvora, A., Gurlebeck, D., Peart, J., Ganal, M., Baker, B., Bonas, U. and Lahaye, T. 2004. The tomato resistance protein Bs4 is a predicted non-nuclear TIR-NB-LRR protein that mediates defense responses to severely truncated derivatives of AvrBs4 and overexpressed AvrBs3. Plant Journal 37:46-60.
van Ooijen, G., van den Burg, H. A., Cornelissen, B. J. C. and Takken, F. L. W. 2007. Structure and function of resistance proteins in solanaceous plants. Ann. Rev. Phytopathol. 45:43-72.

1. An isolated polynucleotide which is selected from the following polynucleotides:
the polynucleotide of SEQ ID No. 1, the polynucleotide of SEQ ID No. 3, the polynucleotide of SEQ ID No. 5, the polynucleotide of SEQ ID No. 7, the polynucleotide of SEQ ID No. 9 or the polynucleotide of SEQ ID No. 11;
a polynucleotide conferring Globodera nematode resistance to plants of the Solanaceae family which is a polynucleotide with at least 80% homology with the polynucleotide of SEQ ID No. 1, the polynucleotide of SEQ ID No. 3, the polynucleotide of SEQ ID No. 5, the polynucleotide of SEQ ID NO. 7, the polynucleotide of SEQ ID No. 9 or the polynucleotide of SEQ ID No. 11;
a polynucleotide coding for the polypeptide of SEQ ID No. 2, the polypeptide of SEQ ID No. 4, the polypeptide of SEQ ID No. 6, the polypeptide of SEQ ID No. 8, the polypeptide of SEQ ID No. 10 or the polypeptide of SEQ ID No. 12; or
a polynucleotide conferring Globodera nematode resistance to plants of the Solanaceae family which is a polynucleotide which is a fragment of a polynucleotide of SEQ ID No. 1, of a polynucleotide of SEQ ID No. 3, of a polynucleotide of SEQ ID No. 5, of a polynucleotide of SEQ ID NO. 7, of a polynucleotide of SEQ ID No. 9 or of a polynucleotide of SEQ ID No. 11.

2. (canceled)

3. The isolated polynucleotide of claim 1, wherein the plants of the Solanaceae family are selected from plants of the Solanum tuberosum L., Solanum phureja, Solanum lycopersicum L., Solanum melongena L. and Nicotiana tabacum species and plants of the genus Capsicum.

4. The isolated polynucleotide of claim 1, wherein the nematodes are selected from Globodera pallida, Globodera rostochiensis, Globodera tabacum ssp. tabacum, ssp. virginiae and ssp. solanacearum, and Globodera mexicana.

5. An expression cassette characterized in that it comprises in the direction of transcription:
a functional promoter in a host organism,
an isolated polynucleotide of claim 1;
a functional termination sequence in the same host organism.

6. The expression cassette of claim 5, wherein the functional promoter in a host organism is selected from CaMV 35S, promoters expressed specifically in roots and the promoter from position 1 to position 1657 of SEQ ID No. 1.

7. A vector comprising a polynucleotide of claim 1.

8. A host cell transformed with a polynucleotide of claim 1.

9. The transformed host cell of claim 8, characterized in that it is selected from plant cells and plant cell protoplasts.

10. A host organism, other than man, transformed with a polynucleotide of claim 1.

11. The transformed host organism, other than man, of claim 10, which is selected from plants, seeds and plant tissue.

12. The transformed host organism, other than man, of claim 10, which the host organism is a plant of the Solanaceae family.

13. The transformed host organism, other than man, of claim 12, wherein the host organism is selected from plants of the species Solanum tuberosum L., Solanum phureja, Solanum lycopersicum L., Solanum melongena L. and Nicotiana tabacum and plants of the genus Capsicum.

14. A plant selected from the species Solanum tuberosum L., Solanum phureja, Solanum lycopersicum L., Solanum melongena L. and Nicotiana tabacum and plants of the genus Capsicum, which comprises a polynucleotide of claim 1.

15. A plant of claim 14, which expresses a polypeptide selected from the polypeptide of SEQ ID No. 2, the polypeptide of SEQ ID No. 4, the polypeptide of SEQ ID No. 6, the polypeptide of SEQ ID No. 8, the polypeptide of SEQ ID No. 10 or the polypeptide of SEQ ID No. 12.

16. A plant selected from crop potatoes, which comprises a polynucleotide of claim 1.

17. A plant selected from crop potatoes of claim 16, which expresses a polypeptide selected from the polypeptide of SEQ ID No. 2, the polypeptide of SEQ ID No. 4, the polypeptide of SEQ ID No. 6, the polypeptide of SEQ ID No. 8, the polypeptide of SEQ ID No. 10 or the polypeptide of SEQ ID No. 12.

18. A method for conferring Globodera nematode resistance to a plant of the Solanaceae family, which comprises transforming the plant with a polynucleotide or an expression cassette or a vector comprising the polynucleotide, wherein the polynucleotide is a polynucleotide of claim 1; and
selecting from the transformed plants, a plant resistant to Globodera nematodes.

19. A method for making a plant of Solanum tuberosum L. subsp. tuberosum, Solanum tuberosum L. subsp. andigena, or Solanum phureja resistant to Globodera nematodes, which comprises the following steps:
introgression of a segment of Solanum sparsipilum genomic DNA comprising the polynucleotide of SEQ ID No. 1 in a plant of Solanum tuberosum L. subsp. tuberosum, Solanum tuberosum L. subsp. andigena, or Solanum phureja; and
selecting from the introgressed plants, a plant resistant to Globodera nematodes, with molecular markers derived from SEQ ID No. 1.

20. A method for detection of nematode-resistant plants comprising using polynucleotide primers or probes derived from SEQ ID No. 1 for detecting nematode-resistant plants, wherein the plants are of the Solanaceae family.
